(19) United States
(12) Patent Application Publication
(10) Pub. No.: US 2002/0076827 A1
(43) Pub. Date: Jun. 20, 2002

(54) Methods for diagnosing thyroid conditions and for monitoring thyroxine therapy

(76) Inventors: Hilton A. Salhanick, Brookline, MA (US); Joachim Hourihan, Dorchester, MA (US)

Correspondence Address: Cooper & Dunham, LLP, 1185 Avenue of the Americas, New York, NY, 10036, US

(21) Appl. No.: 09/915,931

(22) Filed: Jul. 26, 2001

Related U.S. Application Data
(60) Provisional application No. 60/220,894, filed on Jul. 26, 2000.

(51) Int. Cl. G01N 33/566; G01N 33/543

(52) U.S. Cl. 436/501

(57) ABSTRACT

This invention provides a method of diagnosing a thyroid condition in a subject which comprises: determining the concentration of thyroid stimulating hormone in a urine sample by a method which is not a radioimmunoassay; and comparing the concentration of thyroid stimulating hormone with a urinary concentration of thyroid stimulating hormone in a normal subject; wherein: i) a concentration of thyroid stimulating hormone which is higher than the urinary concentration of thyroid stimulating hormone in the normal subject diagnoses hypothyroidism in the subject; and ii) a concentration of thyroid stimulating hormone which is lower than the urinary concentration of thyroid stimulating hormone in the normal subject diagnoses hyperthyroidism in the subject. This invention also proves a method of monitoring thyroxine therapy.

This application is a continuation-in-part of U.S. Provisional Application No. 60/220,894, filed Jul. 26, 2000, the contents of which are hereby incorpoated by reference into this application.

Throughout this application, various publications are referenced by Arabic numerals. Full citations for these publications may be found at the end of the specification immediately preceding the claims. The disclosure of these publications is hereby incorporated by reference into this application to describe more fully the art to which this invention pertains.

### BACKGROUND OF THE INVENTION

The subject invention relates to the development of urine tests for thyroid stimulating hormone (TSH), triiodothyronine (T3), and thyroxine (T4), that will detect abnormal thyroid states and monitor therapy. The invention relates to the validation biochemically and clinically that urinary thyroid stimulating hormone (TSH) is a reliable screening procedure for hypothyroidism and is useful in monitoring therapy. The invention relates to the validation biochemically and clinically that urinary tri-iodothyronine (T3) and/or thyroxine (T4) are reliable screening procedure for hyperthyroidism. The invention relates to the development of methodology for urinary TSH, T3 and T4 tests that can be applied in the physician's office or clinic (i.e. point of care) to yield results within the time interval of a patient's visit. The invention relates to the development of methodologies which utilize application of TSH, T3 and T4 that are simple, inexpensive and conveniently rapid so that the tests can also be performed at home.

Fifty years ago, the diagnosis of disorders of the pituitary-thyroid axis depended upon clinical signs and symptoms, certain clinical assessments such as the basal metabolic rate and comparatively crude laboratory measurements such as protein bound iodine. With the advent of radioactive and immunological methodology, the pituitary and thyroid secretions, thyroid stimulating hormone (TSH), thyroxine (T4) and triiodothronine (T3), were measured sensitively and reliably in blood or serum. Second and third generation immunoassays (radioimmunoassay (RIA) and enzyme-linked immunoassay (ELISA)), facilitated the diagnosis of sub-clinical and forme frust (e.g., atypical) hypothyroidism and hyperthyroidism. Later, advanced assay methodology based upon chemiluminescence, nephelometry and enzyme-linkages resulted in less expensive tests that are automated. Nevertheless, the methods are technologically complicated because both nephelometry and chemiluminescence can be quenched and augmented by alien substances. Immunologic TSH levels can be compromised by immunogenic molecules devoid of biological activity. Clinically, obtaining TSH levels on patients with non-thyroidal illnesses, certain medications and patients just starting on therapy are not dependable [1].

Thyroid disorders are among the most common endocrine disorders. They include but are not limited to underactive and overactive thyroid; benign nodules, thyroid inflammations and malignant cancers. Approximately 12 million Americans are being treated for and even more are afflicted with with thyroid disease. Thyroid diseases occur about five times more often in women than in men. For example, hypothyroidism affects approximately 10% of women who are over 65 years old. Another large risk group for abnormal thyroid function is pregnant women and women in the postpartum period. Approximately 10% of women over 60 years old suffer from subclinical hypothyroidism, which may be considered a condition in which a person does not have overt symptoms but has elevated thryroid stimulating hormone (TSH) concentrations in the blood.

Hypothyroidism may be associated with weight gain, cold intolerance, elevated cholesterol and other lipids, constipation, menstrual irregularities, apathy, depression, and mental impairment. Hypothyroidism is particularly serious in children because it affects growth and mental development. Therefore, all newborns in the United states and most other developed countries are screened for hypothyroidism. Children of women with untreated hypothyroidism during pregnancy have been shown to have lower intelligent quotients than have children who have normal thyroid function. Subclinical hypothyroidism is often associated with elevated cholesterol and triglycerides. In most individuals, the symptoms of hypothyroidism can be alleviated or entirely reversed by appropriate therapy, ascertained at present by measuring TSH levels in blood serum.

Hyperthyroidism may be characterized clinically by weight loss, heat intolerance, insomnia, fatigue, edema (swelling of the legs), increased sweating, muscle weakness, diarrhea, increased pulse rates and palpitations, and in irregular menses in women. Blood glucose may be elevated.

Danese et al [22] analyzed the benefits and costs of routine screening for hypothyroidism by measurement of serum TSH levels at periodic examinations. The study concluded that the cost of the TSH assay is the most influential variable in the model and that the cost effectiveness of large scale screening would be greatest in older women. Therefore, if the cost of screening could be decreased, simple treatment could benefit a large number of people for whom diagnosis is now costly.

The secretion of the thyroidal hormones T4 and T3 from the thyroid gland is regulated by the stimulation of TSH that is secreted by the pituitary gland. In turn, the available (i.e. free) T4 and T3 control the secretion of TSH by the pituitary either directly or indirectly through the hypothalmus and other neural centers. These secretions are “in balance” in a feedback arrangement. If the secretion of T4 is decreased for some reason other than decreased TSH stimulation, the pituitary responds with an increase in TSH secretion to make up the deficiency. If T4 is increased or administered exogenously, the TSH concentration will reflect the inhibition of secretion from the pituitary.

Human thyroid stimulating hormone (TSH) is a glycoprotein of two subunits and it is secreted by the pituitary gland (hypophysis). TSH stimulates the tyroid gland to secrete thyroxine (T4), some of which is converetd to triiodothyroxine (T3). T4 and T3 are snall molecules about twice the size of an average amino acid. Over 99% of T4 and T3 circulate in the blood stream bound to large protein molecules call thyroid binding globulins (TBG) and other protein molecules such as albumin. The bound molecules are biologically inactive. Thus, at present, measurement of T4 and T3 includes protein-bound T4 abd T3 as well as a tiny amount of free hormone. Since only the free hormones are biologically active, the measurement of levels of free, active hormone is fraught with difficulty and the interpretation of the results is often problematic.

The pituitary hormone (TSH) is metabolized very slowly but the thyroid hormones T4 and T3 are metabolized mostly in the liver by transformation to substances with less iodine on the molecule and also by conjugation to the sugar, glucosiduronic acid, and to sulfates. These transformations and conjugations render the molecules biologically inactive and more water-soluble so that they can traverse the renal system and be excreted more rapidly into the urine than the parent substances. Thus, urine contains free T4, free T3, reverse T3 (a relatively inactive isomer of T3), and other metabolic products. In addition, some of the T4, T3 and other products are conjugated to glucosiduronic acid. These conjugates have never been purified and the precise proportions of each are unknown. Furthermore, because urine contains albumin, a portion of T4 and T3 are bound to albumin and possibly to other proteins.

The most important laboratory diagnostic criterion of hypothyroidism has hitherto been the level of TSH in serum. Almost all of the currently available tests for TSH are based upon immunological detection, the most widely used technique being radio-immunassay (RIA). The sensitivity and accuracy of these techniques varies considerably because of differences in methods and instruments. In general, serum TSG levels above 4 to 5 μIU/ml (micro International Units per milliliter) are considered to be elevated over normal. Serum levels of TSH less than 0.4 mIU/ml of serum are considered to be less than normal. Hyperthyroidism based upon excessive secretion of TSH by the pituitary gland is not common and usually low TSH levels imply hyperthyroidism caused by the suppression of the pituitary secretion of TSH by excessive levels of T4 and T3. Thus, definitive diagnosis requires knowledge of both TSH and thyroidal hormones T4 and T3 concentrations.

In addition to diagnosing new disease, another important application is monitoring thyroxine and other therapies of thyroid dysfunction by measuring levels of TSH, T4 and T3 in blood. This monitoring has typically taken place by monitoring serum levels. Individual patients require different dosages of thyroxine so that treatment is finely tuned by modifying dosages in accord with the blood measurements. Optimization of dosages may take months of follow-up. Measurement of these hormones in blood had methodological problems and disadvantages including but are not limited to the following:

(1) discomfort suffered by the subject whose blood is obtained;

(2) The secretion of most hormones is known to be pulsatile. Therefore, a single blood measurement at one point in time is subject to the variability of the pulsatility from minute to minute and also over the course of a day (diurnal variations). Multiple samplings are not acceptable to the patients, not practical for the health provider and are not cost effective.

(3) The thyroid hormones are over 99% bound and inactive; measurements of total hormone concentrations include the 99% of the hormone that is non-active. Measurement of “free hormone” is complicated by the fact that the free and bound are in equilibrium so that concentration is suspect.

(4) Very little free hormone is present (about 10 picograms of free T4 per milliliter of serum) and the differences between normal and abnormal are often small. Thus, technological methods can have large errors especially in the measurement of the free T3 that is the most active of the thyroid hormones and present in least concentrations. Considerable differences are found among commercial methods being used in various hospital and clinical laboratories.

Thus, current methods are costly, inefficient, uncomfortable to the patient and sometimes at the limits of acceptable error. Because of cost, many people with sub-clinical hypothyroidism never get tested.

TSH, T4 and T3 and their conjugates are excreted in urine. In 1935, Hertz and Oastler reported TSH biological activity in urine [8]. Subsequent work demonstrated the presence of TSH, T4 and T3 and by inference based upon hydrolysis, the presence of glucosiduronidate and sulfate conjugates of T4 and T3. Measurements were made initially by extraction of 24 hour collections and more recently, by very sensitive direct radioimmunoassay. The use of urinary thyroidal hormones for individual diagnosis and proper establishment of normal and abnormal levels on large populations has not been done. Of four current textbooks surveyed (such as Braverman and Utiger) no one mentions the possibility of this source. The American Thyroid Associations' diagnostic Guideline recommends the use of serum in spite of its problems. In 1986, one textbook stated that urine measurements of TSH, T4 and T3 had no usefulness. No modern clinical or hospital laboratory systems claim to measure those substances in urine for any purpose.

Measurement of Thyroid Stimulating Hormone and Thyroid Hormones in Blood

Prior to the present invention, the most important diagnostic criteria of thyroid dysfunction are the levels of TSH, T4 and T3 in serum. Because many different methods and instruments are available, the testing sensitivity and accuracy may differ [1]. Generally, TSH levels above 4.5 to 5 milliunits per liter (mU/L) are considered elevated and over 10 mU/L to be pathologically elevated. The low “normal” is 0.2 to 0.3 mU/L. Levels below this may indicate hyperthyroidism in third generation tests.

The metabolism of T4 and T3 is more complex than that of TSH because it involves extra-glandular conversion of T4 to T3 (the active substance), recycling through the hepatic portal system, and over 99% binding to thyroid binding globulin (TBG), and other proteins including transthyretin and albumin. The production of TBG is stimulated by estrogens and diminished by androgens. In addition to T3, other triiodothyronines, especially reversed T3 (RT3) are produced by the liver from T4. Non-thyroidal diseases have a profound effect on the thyroid hormones and TSH [2]. Depending on the severity of the disease, RT3 is elevated, bound and free T4 are decreased as is T3. “For inpatients, only half of TSH concentrations above 20 mU/L and 14% between 6.8 and 20 mU/L were due to primary hypothyroidism; the remainder were transient elevations caused by non-thyroidal illnesses” ([2], page 28). Another variable arises from the portions of T4 and T3 that are glucosiduronidated and sulfurylated by hepatic enzymes and excreted. Thyroxine and triiodothyronine, being small molecules, are excreted in larger quantities than is TSH. The metabolic clearance rates of T4, T3 and RT3 are 1.2, 24 and 111 L/d/70 kg, respectively [3]. For the purposes of this invention, however, it is important to recognize that free T4 and T3 are the most important diagnostic aids in blood. Free T3 cannot be measured directly and reliably in serum ([2], page 25). Of interest, urinary T3 (and possibly T4) are probably more reliable measures of thyroid disease in cases of non-thyroidal illness because the urine levels conform more closely to the true clinical status [4] [5] and others. It is likely that urinary T3 is an accurate measure of free T3 and, therefore, may be a very important indicator of thyroid function.

The normal T4 level in serum is accepted in most laboratories to be 4-11 μg/dL while normal T3 is about 75-175 ng/dL. Over 99% of the hormones are bound. The automated measurements are performed on about 0.1 ml of serum or plasma without extraction or concentration. In addition to the problems cited above, spot serum measurements may not depict the true “steady state” because of diurnal variations [6], minute to minute oscillations, as well as the possibility of laboratory errors inherent in a single determination (as much as 10% in some automated assays). This is especially applicable to measurements on serum from patients with non-thyroid illness. The half-life of TSH is considerably shorter than that of T4 or T3 so that if a patient on medication is being followed, the feedback to the pituitary may not reflect the true thyroidal state unless the interval between taking the drug and measuring the serum levels is taken into consideration. These and other caveats are described extensively by Stockigt [7].

Measurement of Thyroid Stimulating Hormone in Urine

The presence of TSH and the thyroid hormones in urine has generally been overlooked although it has a long history. In fact, not one of three prominent textbooks mentions TSH in urine even once. In 1936, Hertz and Oastler injected 5 ml of urine into a rat daily for five days and determined activity by the histological appearance of the thyroid gland [8]. In 1974, Kuku et al. concentrated urine after dialysis by lyophilization and measured TSH by double antibody RIA [9, 10]. They computed excretion rates of 5.6±0.31 (Mean±S.E.M.) μU/hour in 30 euthyroid patients, 25.1±3.3 μU/hr in 14 hypothyroid patients and 2.6±2.2 μU/hr in 14 hyperthyroid and 7 hypopituitary patients. Correlation between plasma and urine levels was highly significant (P<0.001). No diurnal variations in urinary TSH were detected in contrast to reports on serum [6]. Furthermore, urinary TSH, collected at 3 hour intervals, correlated with blood TSH levels in hypothyroid patients being treated with thyroxine and in patients injected with TSH. Gel filtration profiles of both endogenous and administered TSH showed a large peak in the urine and several minor peaks [9, 10]. Thus, it is likely that urine contains more immunoreactive TSH than biological activity. Although these fragments may actually increase the sensitivity of an immunoassay, they are present in insufficient amounts to have an important effect. Van Herle et al. presented a contrary view because he found little TSH and more fragments [11]. However, their results were not supported by prior or subsequent work. In 1988, Yoshida et al. measured urinary TSH in unconcentrated 24-hour specimens in 10 normal patients, five hypothyroid patients and about 30 with various renal pathologies with an ultra-sensitive immunoradioactive bead kit sensitive to 0.03 μU/ml. His gel filtration profiles confirmed the work of Kuku. In addition, up to 3% albumin, increased concentrations of urea, NaCl, CaCl₂, MgSO₄ and pHs between 5 and 8.5 had no effect on the assay. His normal patients excreted less than 0.08 μU/ml and hypothyroid patients excreted levels above 0.1 μU/ml (with no overlap). (These results are of great interest to us because they coincide with our findings.) TSH excretion was increased in renal patients when the urinary protein concentration exceeded 10 gm/day. The β sub-unit was not detected (as is the case in plasma except for pituitary tumors) but the ′ sub-unit increased with elevated TSH levels and in menopausal women.

Measurement of Thyroxine and Triiodothyronine in Urine

In 1972, Chan et al published the first methods for measuring T4 and T3 in urine [4]. The hormones were measured by RIA on 3 ml aliquots of 24 hour urines after acidification, extraction and concentration. Values for urines collected over 28 consecutive days fell within a very narrow range. Euthyroid patients, both outpatient and hospitalized, and pregnant women had urinary T4 levels of 8.0 to 8.1 (±2.1) μg/day as compared to 2.8±0.9 for hypothyroid and 19.3±8.8 for hyperthyroid (P<0.001 for hypo- and hyperthyroid vs normal). T4 and T3 levels in urine were closely correlated (r=0.863). Mean T3 concentrations for the euthyroid, pregnant, hypothyroid and hyperthyroid patients were 2.9, 3.3, 0.9 and 9.3 μg/day, respectively, with highly significant differences as cited for T4 above. TSH stimulation caused T3 and T4 excretion to double on the second 24-hour specimen.

The work was confirmed and extended by Burke et al who demonstrated that Chan's results were essentially “total” T4 and T3 concentrations and that about 50% was conjugated mostly as glucosiduronidates [5]. Thus, his levels of free hormone were about 50-60% lower than those of Chan. The latter levels were confirmed by Hufner and Hesch [12]. A more detailed study at Mayo Clinic again confirmed Burke's data and showed that the urinary levels of T3 correlated with the plasma levels of T3 and free T4 (r=0.74). Furthermore, their T3 antibody was “specific for T3 and does not recognize a wide variety of T3 and T4 analogs and derivatives” (page 544, [13]).

Our studies indicate that prior conclusions that T4 and T3 are both excreted as glucuronides and sulfates are not accurate. Our data show that T4 is conjugated almost exclusively as a glucuronide and T3 almost excludively as a sulfate.

In 1980, the work was again reconfirmed by Yoshida et al [14]. Euthyroid patients (n=45), hyperthyroid (n=18) and hypothyroid (n=14) patients excreted 0.81, 7.48 and 0.14 μg/day, respectively. Pregnant women excreted levels within the euthyroid range. No levels overlapped other than one hypothyroid patient's level that fell within the euthyroid group. Correlation between urinary T3 and plasma free T3 was 0.97 (r). Their T3 antibody also was specific for T3.

Uses of TSH and T3 Urine Tests

We believe that the optimal urine testing for thyroid states would be parallel tests for TSH and unbound T3. Such tests are useful for: (a) diagnosing thyroid diseases in the setting of a physician's office; (b) screening patients for thyroid dysfunction in the same setting including the exclusion of low risk subjects; (c) screening populations, especially high risk groups; (d) monitoring therapy, particularly thyroid replacement.

Among the “misuses” of serum TSH assays cited by Nicoloff are the screening for hypothalamic-pituitary disorders and transitional states of hypothalamic-pituitary response to acute circulating thyroxine levels, the screening of hospitalized patients with non-thyroidal disease and the measurement of transitional states [1]. These contra-indications may not apply to urinary assessment of thyroid function because urinary T3 measurements appear to yield more reliable information on hospitalized patients with non-thyroidal disease and pregnant women than do the blood analyses (vide supra). Urine analyses of many of the other conditions (e.g., glucocorticoid therapy and therapy with certain drugs that affect binding, etc.) cited by Nicoloff have not been studied yet nor has monitoring during transitional phases of therapy. An inexpensive home test might prove to be useful for this function.

It should be noted that there are various reasons why a urine based test is an improvement over serum based tests. One factor which could impact the measurements could be the presence of substances which either inhibit or stimulate fluorescence. Another factor which could affect the measurements in serum or blood could be the presence of various proteins. Another factor is that much of the T3 in blood is bound to protein (i.e. not free T3). Accordingly, in order to determine the amount of T3, one would need to displace the T3 from the protein. However, it had previously been thought that the T3 in urine was free T3 and accordingly, displacement would not be needed. However, it is now known that most of T3 in urine is also bound to protein and accordingly, displacement may be necessary in order to determine the total amount of T3 in the urine, or a method which measures total T3 is necessary. Accordingly, based on these differences, or perceived differences, it would not have been apparent to take urinary measurements in order to accurately and efficiently diagnose thyroid conditions or to monitor thyroxine therapy.

Epidemiology of Thyroid Disease

The commercial potential for these tests is directly dependent upon the size of the market. Collectively, abnormal thyroid function involves a relatively large number of people in the United States and increases progressively with age. Considering the populations in order of age, at birth, over 4 million babies are now screened for hypothyroidism. Undiagnosed hypothyroidism in pregnant women occurs infrequently (about 2.5/1000) but the consequences for their children may be serious [15]. Furthermore, if this incidence applies to larger populations, over 10,000 babies could be affected annually in the United States. While it is likely that many of the hypothyroid pregnant women have chronic autoimmune thyroiditis, the National Health and Nutrition Examinations Survey III (1988-1994) showed that 6.7% of pregnant women excreted less than 5 μg iodine/dL [16]. This incidence exceeds that of previous surveys by a substantial margin. The overall implication is that approximately 4.5 million pregnant women should be screened. In addition, thyroid dysfunction can be detected in between 4 and 8% of postpartum women [17]. Thyroid nodules have an incidence of 0.1%/year and a clinical prevalence of 4-7% representing about 10 million people in the United States [18]. Even more are detectable radiologically or at autopsy. Of the 275,000 nodules newly detected annually, about 18,000 are malignant. Finally, sex and age are important discriminants. The Whickham Study designed to estimate the frequency of hypothyroidism in a county in England representative of the entire British population found a prevalence of 14/1000 to 19/1000 for women and about 1/1000 for men [19]. The rates increased in women with increasing age but not in men. In the Framingham Study in the United States, 5.9% of women over 60 years of age and 2.3% of men had clearly elevated TSH levels [20]. Another 5.9% had slightly elevated serum levels of TSH. About 15% of women over 65 showed evidence of hypothyroidism [21].

The optimal frequency of testing to monitor thyroxine treatment elicits some debate. Nevertheless, about 10 million patients are being treated with thyroxine annually in the United States and presumably being monitored at least once or twice/year. In this scenario, the patient is seen by the physician, blood is drawn and the patient and physician have another contact after the result is received from the laboratory. A simple, accurate and immediate urine test for both TSH and T3 might yield equivalent, if not better, results and would avoid a second contact.

As reported in the New York Times on Jul. 24, 2001 and in the Wall Street Journal on Jun. 1, 2001, the Food and Drug Administration (FDA) is requiring Abbott Laboratories to submit a new drug application (NDA) for Synthroid, its widely used thyroid medication, because of questions regarding its potency. This has raised the issue regarding whether patients would change medication, which then raises the issue of determining the proper dosage of a new medication for a particular patient since brands differ in their potency. The New York Times article states that switching medication would require several office visits and rounds of blood tests to adjust the dose. There have even been questions regarding whether the potency of particular brands is consistent from batch to batch. Some patients taking thyroxine treatment had been reporting that they were either getting too much or insufficient treatment. These issues reinforce the need for a test which can be performed frequently and possibly even in the patient's home. A serum based assay is not practical since a patient is unlikely to draw his or her own blood. However, a urinary based assay would enable more frequent testing which could even be performed in the home or at the point of care.

Cost-benefit Analysis of TSH Screening

As diagnostic technology becomes more sensitive and more precise, marginal diagnoses can be made. Controversy on diagnosis and more on therapy ensues. Many of the cases cited above fall in the category of sub-clinical hypothyroidism. These patients have chemical evidence of disease-not only in TSH levels but also in certain lipid factors and other physiological parameters. Therapy is clearly controversial because it is possible to cause considerable harm by treating older persons with excessive thyroxine. Nonetheless, the question of screening is independent of therapeutic decisions reached after a diagnosis is made.

In light of the high incidence of sub-clinical hypothyroidism, the benefits and costs of routine screening of serum TSH levels at periodic examinations were analyzed [22]. The basis for recommendation was the rate of progression to overt hypothyroidism (5-26%/year in patients with mild thyroid failure), hypercholesterolemia and quality of life which would be improved by treatment. “The TSH assay cost was the most influential variable in the model. At TSH laboratory “costs varied from $10 to $50, the cost-effectiveness increased from $3,974 to $17,998 per QALY” (page 290) (QALY is quality adjusted life year). The conclusion of this important study was that the cost effectiveness would be greatest in older women and if the cost of assessment could be decreased, it would be desirable to institute a screen for hypothyroidism. Similarly, the Treatment Guidelines proposed by the Standards of Care Committee of the American Thyroid Association recommended screening in elderly patients and others with symptoms including depression and elevated cholesterol [23]. Neither of these evaluations emphasized the pressures of managed care to limit expensive screening tests. A sensitive, convenient and inexpensive screening tool would be useful.

Comparison of Urinary TSH and T3 Tests and Serum Tests

The current procedures for detecting hypothyroidism, diagnosing it and monitoring treatment have many drawbacks:

The measurement of serum TSH requires a venipuncture that is often inconvenient, of concern to some patients, requires professional skill, and, rarely, may have untoward side effects including vaso-vagal syncope or hematoma. In contrast, few problems and no hazards are associated with a single urine collection.

Considerable time delays may occur between obtaining the blood for testing, transport and measurement at a laboratory, informing the patient and instituting therapy. For example, if a physician draws blood for a TSH test at the first visit, there is a delay while the test is performed, and a second visit may be necessary to communicate with the patient and begin therapy. An in-office urine collection may require about 10 minutes of a technician's time (even a lay person can perform the test) and gives an immediate result.

The required clinical and laboratory input for serum measurements is so expensive that many health maintenance organizations and physicians limit these tests to patients with appropriate symptomatology or to patients who are in a high risk category. On the other hand, a urine screen for both TSH and T3 can be made which is more cost effective.

Scientific advantages and disadvantages apply to both methods. A single serum determination allows for error on the part of collection of specimen that may be inaccurate because of occasional difficulties of venipuncture, stability, labeling and other errors in transport of many specimens, and errors in laboratory determination. Significantly, the secretion of hormones varies from minute to minute as well as diurnally and a single point in time may give an erroneous value. An 8 AM sample may be as much as one-third higher than a 2 PM sample [6]. Rarely is the time of last medication of drugs such as thyroxine taken into consideration. Fortunately, there is considerable latitude of values for most purposes but marginal concentrations may be misleading.

On the other hand, the error of diurnal variation involved in the on-site, rapid measurement of a urine specimen may be less. The determination on a single voiding of urine represents a pool over an interval of time that might be as short or as long as the patient and physician determine. Urines can be pooled to represent a 24-hour specimen or collected as a single overnight specimen to represent approximately an 8 hour pool. The serum TSH variability on patients receiving T4 for hypothyroidism is so enormous (0.01 to 20 mU/L) that it is often ignored (FIG. 12-9, P. 230 [6]). Symptomatology for mild dysfunction, especially in those with prior history of abnormal function, is not a very useful guide. Therefore, in the absence of precise guides, and with a desultory feed-back system, infrequent maintenance review is the rule [23].

Serum T3 and T4 levels are influenced by protein binding, entero-hepatic recycling, degradation, and metabolism as well as by excretion. Furthermore, it is likely that most of T4's activity results from peripheral conversion to T3. Patients with non-thyroidal illnesses, pregnant women and those on certain therapies such as glucocorticoids often give misleading results. Conversely, spot urinary measurements have been criticized because levels depend upon all of the above rates of change and renal function. However, neither protein concentration, specific gravity, nor pH has significant effect upon urine measurements of TSH. The concept that creatinine ratios should be used has not been substantiated.

Although both Bayer and Abbott companies propose methods, often indirect, to measure free T3, Kaptein wrote in 1999: “Until free-T3 immunoassay methods with fully characterized analytic performances become available, actual free-T3 levels in sera of nonthyroidal illnesses cannot be determined accurately” [Kaptein, 1999 #29]. Burke, on the other hand, found that T3 measures of thyroid responses are more rapidly observed in urine than in serum [5]. Burke and others cited above confirm the utility of urinary T3 determinations. In fact, urinary T3 may be the most valid laboratory measure of true thyroid state that is available.

Accordingly, the subject invention provides methods and kits which can be performed at the point of care for measuring TSH, T4 and/or T4 and their metabolites in urine to diagnose certain thyroid conditions, such as hyperthyroidism and hypothyroidism, and also to monitor thyroxine therapy, The subject invention is an improvement over the previously described methods in various ways including but not limited to the following: (1) it is a urine based assay in contrast to a serum based assay; (2) it is not a radioimmunoassay and thus, can be performed at the point of care ; (3) it may not require concentrating the urine sample.

### SUMMARY OF THE INVENTION

This invention provides a method of diagnosing a thyroid condition in a subject which comprises: a) obtaining a suitable urine sample from the subject; b) determining the concentration of thyroid stimulating hormone in the sample by a method which is not a radioimmunoassay; and c) comparing the concentration of thyroid stimulating hormone with a urinary concentration of thyroid stimulating hormone in a normal subject; wherein: i) a concentration of thyroid stimulating hormone which is higher than the urinary concentration of thyroid stimulating hormone in the normal subject diagnoses hypothyroidism in the subject; and ii) a concentration of thyroid stimulating hormone which is lower than the urinary concentration of thyroid stimulating hormone in the normal subject diagnoses hyperthyroidism in the subject.

This invention provides a method of diagnosing a thyroid condition in a subject which comprises: a) obtaining a suitable urine sample from the subject; b) determining the concentration of triiodothyronine in the sample by a method which is not a radioimmunoassay; and c) comparing the concentration of triiodothyronine with a urinary concentration of triiodothyronine in a normal subject; wherein i) a concentration of triiodothyronine which is lower than the urinary concentration of triiodothyronine in the normal subject diagnoses hypothyroidism in the subject; and ii) a concentration of triiodothyronine which is higher than the urinary concentration of triiodothyronine in the normal subject diagnoses hyperthyroidism in the subject.

This invention provides a method of diagnosing a thyroid condition in a subject which comprises: a) obtaining a suitable urine sample from the subject; b) determining the concentration of triiodothyronine-sulfate in the sample by a method which is not a radioimmunoassay; and c) comparing the concentration of triiodothyroninesulfate with a urinary concentration of triiodothyroninesulfate in a normal subject; wherein i) a concentration of triiodothyronine-sulfate which is lower than the urinary concentration of triiodothyronine-sulfate in the normal subject diagnoses hypothyroidism in the subject; and ii) a concentration of triiodothyronine-sulfate which is higher than the urinary concentration of triiodothyronine-sulfate in the normal subject diagnoses hyperthyroidism in the subject.

This invention provides a method of diagnosing a thyroid condition in a subject which comprises: a) obtaining a suitable urine sample from the subject; b) determining the concentration of thyroxine present in the sample by a method which is not a radioimmunoassay; c) comparing the concentration of thyroxine with a urinary concentration of thyroxine in a normal subject; wherein i) a concentration of thyroxine which is lower than the concentration of thyroxine in the normal subject diagnoses hypothyroidism in the subject; and ii) a concentration of thyroxine which is higher than the urinary concentration of thyroxine in the normal subject diagnoses hyperthyroidism in the subject.

This invention provides a method of diagnosing a thyroid condition in a subject which comprises: a) obtaining a suitable urine sample from the subject; b) determining the concentration of thyroxine-glucuronide present in the sample by a method which is not a radioimmunoassay; c) comparing the concentration of thyroxine-glucuronide with a urinary concentration of thyroxine-glucuronide in a normal subject; wherein i) a concentration of thyroxine-glucuronide which is lower than the concentration of thyroxine-glucuronide in the normal subject diagnoses hypothyroidism in the subject; and ii) a concentration of thyroxine-glucuronide which is higher than the urinary concentration of thyroxine-glucuronide in the normal subject diagnoses hyperthyroidism in the subject.

This invention provides a method of diagnosing a thyroid condition in a subject which comprises: a) obtaining a suitable urine sample from the subject; b) determining the concentration of thyroid stimulating hormone and the concentration of triiodothyronine in the sample by a method which is not a radioimmunoassay; c) comparing the concentration of thyroid stimulating hormone with a urinary concentration of thyroid stimulating hormone in a normal subject and comparing the concentration of triiodothyronine with a urinary concentration of triiodothyronine in a normal subject; wherein i) concentration of thyroid stimulating hormone which is higher than the urinary concentration of thyroid stimulating hormone in the normal subject, and a concentration of triiodothyronine which is lower than the urinary concentration of triiodothyronine in the normal subject, diagnoses hypothyroidism in the subject; and ii) concentration of thyroid stimulating hormone which is lower than the urinary concentration of thyroid stimulating hormone present in the normal subject, and a concentration of triiodothyronine which is higher than the urinary concentration of triiodothyronine in the normal subject, diagnoses hyperthyroidism in the subject. This invention provides a method of diagnosing a thyroid condition in a subject which comprises: a) obtaining a suitable urine sample from the subject; b) determining the concentration of thyroid stimulating hormone and the concentration of thyroxine in the sample by a method which is not a radioimmunoassay; c) comparing the concentration of thyroid stimulating hormone with a urinary concentration of thyroid stimulating hormone in a normal subject and comparing the concentration of thyroxine with a urinary concentration of thyroxine in a normal subject; wherein i) a concentration of thyroid stimulating hormone which is higher than the urinary concentration of thyroid stimulating hormone in a normal subject, and a concentration of thyroxine which is lower than the urinary concentration of thyroxine in a normal subject, diagnoses hypothyroidism in the subject; and ii) a concentration of thyroid stimulating hormone which is lower than the urinary concentration of thyroid stimulating hormone in a normal subject, and a concentration of thyroxine which is higher than the urinary concentration of thyroxine in a normal subject, diagnoses hyperthyroidism in the subject.

This invention provides a method of determining whether a subject being treated with thyroxine is receiving a proper dosage of thyroxine which comprises: a) obtaining a suitable urine sample from the subject; b) determining the concentration of thyroid stimulating hormone in the sample by a method which is not a radioimmunoassay; and c) comparing the concentration of thyroid stimulating hormone with a urinary concentration of thyroid stimulating hormone in a normal subject; wherein a concentration of thyroid stimulating hormone which is higher or lower than the urinary concentration of thyroid stimulating hormone in a normal subject indicates that the subject is not receiving the proper dosage of thyroxine.

This invention provides a method of determining whether a subject being treated with thyroxine is receiving a proper dosage of thyroxine which comprises: a) obtaining a suitable urine sample from the subject; b) determining the concentration of triiodothyronine in the sample by a method which is not a radioimmunoassay; and c) comparing the concentration of triiodothyronine with a urinary concentration of triiodothyronine in a normal subject; wherein a concentration of triiodothyronine which is higher or lower than the urinary concentration of triiodothyronine in a normal subject indicates that the subject is not receiving the proper dosage of thyroxine.

This invention provides a method of determining whether a subject being treated with thyroxine is receiving a proper dosage of thyroxine which comprises: a) obtaining a suitable urine sample from the subject; b) determining the concentration of thyroxine in the sample by a method which is not a radioimmunoassay; c) comparing the concentration of thyroxine with a urinary concentration of thyroxine in a normal subject; wherein a concentration of thyroxine which is higher or lower than the urinary concentration of thyroxine in a normal subject indicates that the subject is not receiving the proper dosage of thyroxine.

### BRIEF DESCRIPTION OF THE FIGURES

FIG. 1: Inter-Assay (Abbott Standards and BioDiagnostic Standard). The BioDx urinary standards have precision coefficients similiar to Abbott's serum standards.

FIG. 2: accuracy experiment, wherein the data are plotted at total range (0 to 42) (Panel A) and lower range (0 to 2) (Panel B).

FIG. 3: stability of serum and urine standards at room temperature.

FIG. 4: effect of specific gravity (Panel A) and pH (Panel B) on urine TSH concentrations.

FIG. 5: analysis of Serum and Urine TSH Levels

FIG. 6: Inter-assay precision data for untreated, thyroxine treated, and tapazole treated subject.

FIG. 7: Spectroscopic Scan of Pure Triiodothyronine: 100 μg of thyroxine, dissolved in borate buffer, pH 7.43 in a one ml cuvette with one cm light path was scanned between the shown wavelengths in a Beckman DU 600 Spectrophotometer. The absorption maxima are shown in the insert.

FIG. 8: Spectroscopic Scan of Purified Triiodothyronine Sulfate: 100 μg of thyroxine-sulfate, dissolved in borate buffer, pH 7.43 in a one ml cuvette with one cm light path was scanned between the shown wavelengths in a Beckman DU 600 Spectrophotometer. The absorption maxima are shown in the insert.

### DETAILED DESCRIPTION OF THE INVENTION

As used herein, Thyroid Stimulating Hormone may also be referred to as TSH. As discussed in the Merck Index (12^{th} Edition, published by Merck Research Laboratories of Merck & Co. 1996) TSH may also be referred to as Thyrotropin, thyrotropic hormone, thyreotrophic hormone, throid-stimulating hormone, TTH, dermathycin, and thytropar.

As used herein, triiodothyronine may also be referred to as T3. As discussed in the Merck Index, T3 may also be referred to as Liothyroxine, and the following chemical formulas are provided: O-(4-hydroxy-3-iodophenyl)-3,5-diiodo-L-tyrosine; L-3[4-(4-hydroxy-3-iodophenoxy)-3,5-di-iodophenyl]alanine; 4-(3-iodo-4-hydroxyphenoxy)-3,5-di-iodophenylalanine; 3,5,3′-triiodothyroxine; T-3; C₁₅, H₁₂, I₃—NO₄.

As used herein, thyroxine may also be referred to as T4. As discussed in the Merck Index, T4 may have the following formulas: O-(4-hydroxy-3,5-didiodophenyl)-3,5-diiodotyrosine; 3-[4-(4-hydroxy-3,5-diiodophenoxy)-3,5-diiodophenyl]alanine; β-[(3,5-diiodo-4-hydroxy-4-hydroxyphenoxy)-3,5-diiodophenyl]a;anine; 3,5,3′,5′-tetraiodothyroxine; C₁₅, H₁₁, I₄NO₄.

As used herein “IU” refers to international units and μIU referes to micro international units and is based on the WHO Standard 80/558 units. Accordingly, μIU is based on the WHO Reference standard 80/558. The standard wa stested in urine stripped of TSH as well as in borate buffer and in Abbott Laboratories “zero calibrator.” Such testing medium is urine buffered with 0.01 borate buffer. Such standard was determined as the Second International Reference Preparation of Thyroid Stimulating Hormone, Human, for Immunoassay (2^{nd} IRP, hTSH, for immunoassay, code no. 80/558, established 1983) by the National Institute for Biological Standards and Control, located at Bianche Lane, South Mimms, Potters Bar, Hertfordshire EN6 3QG, United Kingdom. Such calibration and establishment is described in Gaines-Das et al. (1985), Journal of Endocrinology 104:367-379.

This invention provides a method of diagnosing a thyroid condition in a subject which comprises: a) obtaining a suitable urine sample from the subject; b) determining the concentration of thyroid stimulating hormone in the sample by a method which is not a radioimmunoassay; and c) comparing the concentration of thyroid stimulating hormone with a urinary concentration of thyroid stimulating hormone in a normal subject; wherein: i) a concentration of thyroid stimulating hormone which is higher than the urinary concentration of thyroid stimulating hormone in the normal subject diagnoses hypothyroidism in the subject; and ii) a concentration of thyroid stimulating hormone which is lower than the urinary concentration of thyroid stimulating hormone in the normal subject diagnoses hyperthyroidism in the subject.

In one embodiment of the above method, step (b) comprises: (1) contacting an agent capable of binding to thyroid stimulating hormone with the urine sample so as to bind thyroid stimulating hormone which is present in the sample to the immobilized agent; (2) removing unbound urine sample; (3) contacting the bound thyroid stimulating hormone with a detectable agent capable of binding to thyroid stimulating hormone so as to bind the detectable agent to the bound thyroid stimulating hormone; (4) removing unbound detectable agent; and (5) determining the amount of detectable agent which is bound to the thyroid stimulating hormone, thereby determining the amount of thyroid stimulating hormone in the urine sample.

In one embodiment of the above method, the agent capable of binding to thyroid stimulating hormone of step (1) is an antibody which binds to thyroid stimulating hormone.

In one embodiment of the above method, the detectable agent is an antibody which binds to an epitope on thyroid stimulating hormone which differs from the epitope to which the immobilized agent of step (1) binds. In one embodiment of the above method, the detectable agent is labeled with a detectable marker.

In one embodiment of the above method, a concentration of thyroid stimulating hormone greater than 0.35 μIU/ml diagnoses hypothyroidism in the subject. In one embodiment of the above method, a concentration of thyroid stimulating hormone less than 0.04 μIU/ml diagnoses hyperthyroidism in the subject.

As used herein, a normal subject may also be referred to as one is euthyroid. A normal subject is one who has urinary concentrations of particular substances (such as TSH, T3, T4 and other derivatives) which fall in the range of a subject who does not have a thyroid condition (such as hypothyroidism or hyperthyroidism). One skilled in the art can determine the normal urinary concentrations by assaying individuals already known to be normal and individuals already known to have a thyroid condition, and thereby determine the range for a normal individual. In one embodiment, the subject who is already known to be either normal or as having the thyroid condition may be one who was previously tested through a serum sample.

This invention provides a method of diagnosing a thyroid condition in a subject which comprises: a) obtaining a suitable urine sample from the subject; b) determining the concentration of triiodothyronine in the sample by a method which is not a radioimmunoassay; and c) comparing the concentration of triiodothyronine with a urinary concentration of triiodothyronine in a normal subject; wherein i) a concentration of triiodothyronine which is lower than the urinary concentration of triiodothyronine in the normal subject diagnoses hypothyroidism in the subject; and ii) a concentration of triiodothyronine which is higher than the urinary concentration of triiodothyronine in the normal subject diagnoses hyperthyroidism in the subject.

In one embodiment of the above method, step (b) comprises: (1) contacting an agent capable of binding to triiodothyronine with a pre-determined amount of detectable triiodothyronine and the urine sample, so as to form a complex between the agent and (i) the detectable triiodothyronine or (ii) the triiodothyronine present in the urine sample; (2) determining the amount of detectable triiodothyronine which is bound to the agent, wherein the difference between the pre-determined amount of detectable triiodothyronine and the amount of detectable triiodothyronine which is bound indicates the amount of triiodothyronine present in the urine sample.

In one embodiment of the above method, step (b) comprises: (1) contacting an agent capable of binding to triiodothyronine with a pre-determined amount of detectable triiodothyronine and the urine sample, so as to form a complex between the agent and (i) the detectable triiodothyronine or (ii) the triiodothyronine present in the urine sample; (2) determining the amount of detectable triiodothyronine which is not bound to the immobilized agent, thereby determining the amount of triiodothyronine present in the urine sample.

In one embodiment of the above method, the agent of step (1) which is capable of binding to triiodothyronine is an antibody. In one embodiment of the above method, the agent of step (1) which is capable of binding to triiodothyronine is a triiodothyronine receptor. In one embodiment of the above method, the detectable triiodothyronine is labeled with a detectable marker.

In one embodiment of the above method, a concentration of triiodothyronine which is less than 0.3 ng/ml diagnoses hypothyroidism in the subject. In one embodiment of the above method, a concentration of triiodothyronine which is greater than 1.5 ng/ml diagnoses hyperthyroidism in the subject.

This invention provides a method of diagnosing a thyroid condition in a subject which comprises: a) obtaining a suitable urine sample from the subject; b) determining the concentration of triiodothyronine-sulfate in the sample by a method which is not a radioimmunoassay; and c) comparing the concentration of triiodothyroninesulfate with a urinary concentration of triiodothyroninesulfate in a normal subject; wherein i) a concentration of triiodothyronine-sulfate which is lower than the urinary concentration of triiodothyronine-sulfate in the normal subject diagnoses hypothyroidism in the subject; and ii) a concentration of triiodothyronine-sulfate which is higher than the urinary concentration of triiodothyronine-sulfate in the normal subject diagnoses hyperthyroidism in the subject.

In one embodiment of the above method, step (b) comprises: (1) contacting an agent capable of binding to triiodothyronine-sulfate with a pre-determined amount of detectable triiodothyronine-sulfate and the urine sample, so as to form a complex between the agent and (i) the detectable triiodothyronine-sulfate or (ii) the triiodothyronine-sulfate present in the urine sample; (2) determining the amount of detectable triiodothyroninesulfate which is bound to the agent, wherein the difference between the pre-determined amount of detectable triiodothyronine-sulfate and the amount of detectable triiodothyronine-sulfate which is bound indicates the amount of triiodothyronine-sulfate present in the urine sample.

In one embodiment of the above method, step (b) comprises: (1) contacting an agent capable of binding to triiodothyronine-sulfate with a pre-determined amount of detectable triiodothyronine-sulfate and the urine sample, so as to form a complex between the agent and (i) the detectable triiodothyronine-sulfate or (ii) the triiodothyronine-sulfate present in the urine sample; (2) determining the amount of detectable triiodothyroninesulfate which is not bound to the agent, thereby determining the amount of triiodothyronine-sulfate present in the urine sample.

In one embodiment of the above method, the agent of step (1) which is capable of binding to triiodothyroninesulfate is an antibody. In one embodiment of the above method, the immobilized agent of step (1) which is capable of binding to triiodothyronine-sulfate is a triiodothyronine receptor. In one embodiment of the above method, the detectable triiodothyronine-sulfate is labeled with a detectable marker.

In one embodiment of the above method, a concentration of triiodothyronine-sulfate which is lower than 0.1 ng/ml diagnoses hypothyroidism in the subject. In one embodiment of the above method, a concentration of triiodothyronine-sulfate which is higher than 0.5 ng/ml diagnoses hyperthyroidism in the subject.

This invention provides a method of diagnosing a thyroid condition in a subject which comprises: a) obtaining a suitable urine sample from the subject; b) determining the concentration of thyroxine present in the sample by a method which is not a radioimmunoassay; c) comparing the concentration of thyroxine with a urinary concentration of thyroxine in a normal subject; wherein i) a concentration of thyroxine which is lower than the concentration of thyroxine in the normal subject diagnoses hypothyroidism in the subject; and ii) a concentration of thyroxine which is higher than the urinary concentration of thyroxine in the normal subject diagnoses hyperthyroidism in the subject.

In one embodiment of the above method, step (b) comprises: (1) contacting an agent capable of binding to thyroxine with a pre-determined amount of detectable thyroxine and the urine sample, so as to form a complex between the agent and (i) the detectable thyroxine or (ii) the thyroxine present in the urine sample; (2) determining the amount of detectable thyroxine which is bound to the agent, wherein the difference between the pre-determined amount of detectable thyroxine and the amount of detectable thyroxine which is bound indicates the amount of thyroxine present in the urine sample.

In one embodiment of the above method, step (b) comprises: (1) contacting an agent capable of binding to thyroxine with a pre-determined amount of detectable thyroxine and the urine sample, so as to form a complex between the agent and (i) the detectable thyroxine or (ii) the thyroxine present in the urine sample; (2) determining the amount of detectable thyroxine which is not bound to the agent, thereby determining the amount of thyroxine present in the urine sample.

In one embodiment of the above method, the agent of step (1) which is capable of binding to thyroxine is an antibody. In one embodiment of the above method, the immobilized agent of step (1) which is capable of binding to thyroxine is a thyroxine receptor. In one embodiment of the above method, the detectable thyroxine is labeled with a detectable marker.

In one embodiment of the above method, a concentration of thyroxine which is lower than 0.3 ng/ml diagnoses hypothyroidism in the subject. In one embodiment of the above method, a concentration of thyroxine which is higher than 1.5 ng/ml diagnoses hyperthyroidism in the subject.

This invention provides a method of diagnosing a thyroid condition in a subject which comprises: a) obtaining a suitable urine sample from the subject; b) determining the concentration of thyroxine-glucuronide present in the sample by a method which is not a radioimmunoassay; c) comparing the concentration of thyroxine-glucuronide with a urinary concentration of thyroxine-glucuronide in a normal subject; wherein i) a concentration of thyroxine-glucuronide which is lower than the concentration of thyroxine-glucuronide in the normal subject diagnoses hypothyroidism in the subject; and ii) a concentration of thyroxine-glucuronide which is higher than the urinary concentration of thyroxine-glucuronide in the normal subject diagnoses hyperthyroidism in the subject.

In one embodiment of the above method, step (b) comprises: (1) contacting an agent capable of binding to thyroxine-glucuronide with a pre-determined amount of detectable thyroxine-glucuronide and the urine sample, so as to form a complex between the agent and (i) the detectable thyroxine-glucuronide or (ii) the thyroxine-glucuronide present in the urine sample; (2) determining the amount of detectable thyroxine-glucuronide which is bound to the agent, wherein the difference between the pre-determined amount of detectable thyroxine-glucuronide and the amount of detectable thyroxine-glucuronide which is bound indicates the amount of thyroxine-glucuronide present in the urine sample.

In one embodiment of the above method, step (b) comprises: (1) contacting an agent capable of binding to thyroxine-glucuronide with a pre-determined amount of detectable thyroxine-glucuronide and the urine sample, so as to form a complex between the agent and (i) the detectable thyroxine-glucuronide or (ii) the thyroxine-glucuronide present in the urine sample; (2) determining the amount of detectable thyroxine-glucuronide which is not bound to the agent, thereby determining the amount of thyroxine-glucuronide present in the urine sample.

In one embodiment of the above method, the agent of step (1) which is capable of binding to thyroxine-glucuronide is an antibody. In one embodiment of the above method, the agent of step (1) which is capable of binding to thyroxine-glucuronide is a thyroxine receptor. In one embodiment of the above method, the detectable thyroxine-glucuronide is labeled with a detectable marker.

In one embodiment of the above method, a concentration of thyroxine-glucuronide which is lower than 0.1 ng/ml diagnoses hypothyroidism in the subject. In one embodiment of the above method, a concentration of thyroxine-glucuronide which is higher than 0.5 ng/ml diagnoses hyperthyroidism in the subject.

This invention provides a method of diagnosing a thyroid condition in a subject which comprises: a) obtaining a suitable urine sample from the subject; b) determining the concentration of thyroid stimulating hormone and the concentration of triiodothyronine in the sample by a method which is not a radioimmunoassay; c) comparing the concentration of thyroid stimulating hormone with a urinary concentration of thyroid stimulating hormone in a normal subject and comparing the concentration of triiodothyronine with a urinary concentration of triiodothyronine in a normal subject; wherein i) concentration of thyroid stimulating hormone which is higher than the urinary concentration of thyroid stimulating hormone in the normal subject, and a concentration of triiodothyronine which is lower than the urinary concentration of triiodothyronine in the normal subject, diagnoses hypothyroidism in the subject; and ii) concentration of thyroid stimulating hormone which is lower than the urinary concentration of thyroid stimulating hormone present in the normal subject, and a concentration of triiodothyronine which is higher than the urinary concentration of triiodothyronine in the normal subject, diagnoses hyperthyroidism in the subject. In one embodiment of the above method, step (b) comprises: (1) contacting an agent capable of binding to thyroid stimulating hormone with the urine sample so as to bind thyroid stimulating hormone which is present in the sample to the agent; (2) removing unbound urine sample; (3) contacting the bound thyroid stimulating hormone with a detectable agent capable of binding to thyroid stimulating hormone so as to bind the detectable agent to the bound thyroid stimulating hormone; (4) removing unbound detectable agent; and (5) determining the amount of detectable agent which is bound to the thyroid stimulating hormone, thereby determining the amount of thyroid stimulating hormone in the urine sample.

In one embodiment of the above method, the agent capable of binding to thyroid stimulating hormone of step (1) is an antibody which binds to thyroid stimulating hormone. In one embodiment of the above method, the agent capable of binding to thyroid stimulating hormone of step (1) is a receptor which binds to thyroid stimulating hormone. In one embodiment of the above method, the detectable agent is an antibody which binds to an epitope on thyroid stimulating hormone which differs from the epitope to which the immobilized agent of step (1) binds. In one embodiment of the above method, the detectable agent is labeled with a detectable marker.

In one embodiment of the above method, step (b) comprises: (1) contacting an agent capable of binding to triiodothyronine with a pre-determined amount of detectable triiodothyronine and the urine sample, so as to form a complex between the agent and (i) the detectable triiodothyronine or (ii) the triiodothyronine present in the urine sample; (2) determining the amount of detectable triiodothyronine which is bound to the agent, wherein the difference between the pre-determined amount of detectable triiodothyronine and the amount of detectable triiodothyronine which is bound indicates the amount of triiodothyronine present in the urine sample.

In one embodiment of the above method, step (b) comprises: (1) contacting an agent capable of binding to triiodothyronine with a pre-determined amount of detectable triiodothyronine and the urine sample, so as to form a complex between the agent and (i) the detectable triiodothyronine or (ii) the triiodothyronine present in the urine sample; (2) determining the amount of detectable triiodothyronine which is not bound to the immobilized agent, thereby determining the amount of triiodothyronine present in the urine sample.

In one embodiment of the above method, the agent capable of binding to thyroid stimulating hormone of step (1) which is capable of binding to triiodothyronine is an antibody. In one embodiment of the above method, the agent capable of binding to thyroid stimulating hormone of step (1) which is capable of binding to triiodothyronine is a triiodothyronine receptor. In one embodiment of the above method, the detectable triiodothyronine is labeled with a detectable marker.

In one embodiment of the above method, a concentration of thyroid stimulating hormone greater than 0.35 μIU/ml and a concentration of triiodothyronine greater then 1.5 ng/ml diagnoses hypothyroidism in the subject. In one embodiment of the above method, a concentration of thyroid stimulating hormone less than 0.04 μIU/ml and a concentration of triiodothyronine less than 0.3 ng/ml diagnoses hyperthyroidism in the subject.

This invention provides a method of diagnosing a thyroid condition in a subject which comprises: a) obtaining a suitable urine sample from the subject; b) determining the concentration of thyroid stimulating hormone and the concentration of thyroxine in the sample by a method which is not a radioimmunoassay; c) comparing the concentration of thyroid stimulating hormone with a urinary concentration of thyroid stimulating hormone in a normal subject and comparing the concentration of thyroxine with a urinary concentration of thyroxine in a normal subject; wherein i) a concentration of thyroid stimulating hormone which is higher than the urinary concentration of thyroid stimulating hormone in a normal subject, and a concentration of thyroxine which is lower than the urinary concentration of thyroxine in a normal subject, diagnoses hypothyroidism in the subject; and ii) a concentration of thyroid stimulating hormone which is lower than the urinary concentration of thyroid stimulating hormone in a normal subject, and a concentration of thyroxine which is higher than the urinary concentration of thyroxine in a normal subject, diagnoses hyperthyroidism in the subject.

In one embodiment of the above method, step (b) comprises: (1) contacting an agent capable of binding to thyroid stimulating hormone with the urine sample so as to bind thyroid stimulating hormone which is present in the sample to the agent; (2) removing unbound urine sample; (3) contacting the bound thyroid stimulating hormone with a detectable agent capable of binding to thyroid stimulating hormone so as to bind the detectable agent to the bound thyroid stimulating hormone; (4) removing unbound detectable agent; and (5) determining the amount of detectable agent which is bound to the thyroid stimulating hormone, thereby determining the amount of thyroid stimulating hormone in the urine sample.

In one embodiment of the above method, the agent capable of binding to thyroid stimulating hormone of step (1) is an antibody which binds to thyroid stimulating hormone. In one embodiment of the above method, the detectable agent is an antibody which binds to an epitope on thyroid stimulating hormone which differs from the epitope to which the immobilized agent of step (1) binds. In one embodiment of the above method, the agent capable of binding to thyroid stimulating hormone of step (1) is a receptor which binds to thyroid stimulating hormone. In one embodiment of the above method, the detectable agent is labeled with a detectable marker.

In one embodiment of the above method, step (b) comprises: (1) contacting an agent capable of binding to thyroxine with a pre-determined amount of detectable thyroxine and the urine sample, so as to form a complex between the agent and (i) the detectable thyroxine or (ii) the thyroxine present in the urine sample; (2) determining the amount of detectable thyroxine which is bound to the agent, wherein the difference between the pre-determined amount of detectable thyroxine and the amount of detectable thyroxine which is bound indicates the amount of thyroxine present in the urine sample.

In one embodiment of the above method, step (b) comprises: (1) contacting an agent capable of binding to thyroxine with a pre-determined amount of detectable thyroxine and the urine sample, so as to form a complex between the agent and (i) the detectable thyroxine or (ii) the thyroxine present in the urine sample; (2) determining the amount of detectable thyroxine which is not bound to the agent, thereby determining the amount of thyroxine present in the urine sample.

In one embodiment of the above method, the agent of step (1) which is capable of binding to thyroxine is an antibody. In one embodiment of the above method, the agent of step (1) which is capable of binding to thyroxine is a thyroxine receptor. In one embodiment of the above method, the detectable thyroxine is labeled with a detectable marker.

In one embodiment of the above method, a concentration of thyroid stimulating hormone greater than 0.35 μIU/ml and a concentration of thyroxine greater then 1.5 ng/ml diagnoses hypothyroidism in the subject. In one embodiment of the above method, a concentration of thyroid stimulating hormone less than 0.04 μIU/ml and a concentration of thyroxine less than 0.3 ng/ml diagnoses hyperthyroidism in the subject.

This invention provides a method of determining whether a subject being treated with thyroxine is receiving a proper dosage of thyroxine which comprises: a) obtaining a suitable urine sample from the subject; b) determining the concentration of thyroid stimulating hormone in the sample by a method which is not a radioimmunoassay; and c) comparing the concentration of thyroid stimulating hormone with a urinary concentration of thyroid stimulating hormone in a normal subject; wherein a concentration of thyroid stimulating hormone which is higher or lower than the urinary concentration of thyroid stimulating hormone in a normal subject indicates that the subject is not receiving the proper dosage of thyroxine.

In one embodiment of the above method, step (b) comprises: (1) contacting an agent capable of binding to thyroid stimulating hormone with the urine sample so as to bind thyroid stimulating hormone which is present in the sample to the agent; (2) removing unbound urine sample; (3) contacting the bound thyroid stimulating hormone with a detectable agent capable of binding to thyroid stimulating hormone so as to bind the detectable agent to the bound thyroid stimulating hormone; (4) removing unbound detectable agent; and (5) determining the amount of detectable agent which is bound to the thyroid stimulating hormone, thereby determining the amount of thyroid stimulating hormone in the urine sample.

In one embodiment of the above method, the agent of step (1) is an antibody which binds to thyroid stimulating hormone. In one embodiment of the above method, the detectable agent is an antibody which binds to an epitope on thyroid stimulating hormone which differs from the epitope to which the agent of step (1) binds. In one embodiment of the above method, the agent of step (1) which is capable of binding to thyroid stimulating hormone is a thyroid stimulating hormone receptor. In one embodiment of the above method, the detectable agent is labeled with a detectable marker.

In one embodiment of the above method, a concentration of thyroid stimulating hormone which is higher than 0.35 μIU/ml or a concentration of thyroid stimulating hormone which is lower than 0.04 μIU/ml indicates that the subject is not receiving the proper dosage of thyroxine. This invention provides a method of determining whether a subject being treated with thyroxine is receiving a proper dosage of thyroxine which comprises: a) obtaining a suitable urine sample from the subject; b) determining the concentration of triiodothyronine in the sample by a method which is not a radioimmunoassay; and c) comparing the concentration of triiodothyronine with a urinary concentration of triiodothyronine in a normal subject; wherein a concentration of triiodothyronine which is higher or lower than the urinary concentration of triiodothyronine in a normal subject indicates that the subject is not receiving the proper dosage of thyroxine.

In one embodiment of the above method, step (b) comprises: (1) contacting an agent capable of binding to triiodothyronine with a pre-determined amount of detectable triiodothyronine and the urine sample, so as to form a complex between the agent and (i) the detectable triiodothyronine or (ii) the triiodothyronine present in the urine sample; and (2) determining the amount of detectable triiodothyronine which is bound to the agent, wherein the difference between the pre-determined amount of detectable triiodothyronine and the amount of detectable triiodothyronine which is bound indicates the amount of triiodothyronine present in the urine sample.

In one embodiment of the above method, step (b) comprises: (1) contacting an agent capable of binding to triiodothyronine with a pre-determined amount of detectable triiodothyronine and the urine sample, so as to form a complex between the agent and (i) the detectable triiodothyronine or (ii) the triiodothyronine present in the urine sample; (2) determining the amount of detectable triiodothyronine which is not bound to the agent, thereby determining the amount of triiodothyronine present in the urine sample.

In one embodiment of the above method, the agent of step (1) which is capable of binding to triiodothyronine is an antibody. In one embodiment of the above method, the agent of step (1) which is capable of binding to triiodothyronine is a triiodothyronine receptor. In one embodiment of the above method, the detectable triiodothyronine is labeled with a detectable marker.

In one embodiment of the above method, a concentration of triiodothyronine which is lower than 0.3 ng/ml or a concentration of triiodothyronine which is higher than 1.5 ng/ml indicates that the subject is not receiving the proper dosage of thyroxine.

This invention provides a method of determining whether a subject being treated with thyroxine is receiving a proper dosage of thyroxine which comprises: a) obtaining a suitable urine sample from the subject; b) determining the concentration of thyroxine in the sample by a method which is not a radioimmunoassay; c) comparing the concentration of thyroxine with a urinary concentration of thyroxine in a normal subject; wherein a concentration of thyroxine which is higher or lower than the urinary concentration of thyroxine in a normal subject indicates that the subject is not receiving the proper dosage of thyroxine.

In one embodiment of the above method, step (b) comprises: (1) contacting an agent capable of binding to thyroxine with a pre-determined amount of detectable thyroxine and the urine sample, so as to form a complex between the agent and (i) the detectable thyroxine or (ii) the thyroxine present in the urine sample; (2) determining the amount of detectable thyroxine which is bound to the agent, wherein the difference between the pre-determined amount of detectable thyroxine and the amount of detectable thyroxine which is bound indicates the amount of thyroxine present in the urine sample.

In one embodiment of the above method, step (b) comprises: (1) contacting an agent capable of binding to thyroxine with a pre-determined amount of detectable thyroxine and the urine sample, so as to form a complex between the immobilized agent and (i) the detectable thyroxine or (ii) the thyroxine present in the urine sample; (2) determining the amount of detectable thyroxine which is not bound to the agent, thereby determining the amount of thyroxine present in the urine sample.

In one embodiment of the above method, the agent of step (1) which is capable of binding to thyroxine is an antibody. In one embodiment of the above method, the agent of step (1) which is capable of binding to thyroxine is a thyroxine receptor. In one embodiment of the above method, the detectable thyroxine is labeled with a detectable marker.

In one embodiment of the above method, a concentration of thyroxine which is lower than 0.3 ng/ml or a concentration of thyroxine which is higher than 1.5 ng/ml indicates that the subject is not receiving the proper dosage of thyroxine.

This invention provides modifications of the methods described herein, such as a method of diagnosing a thyroid condition in a subject which comprises: a) obtaining a suitable urine sample from the subject; b) comparing the concentration of any one or any combination of one or more of the compounds described herein (such as TSH, T3, T4, T3-sulfate and T4-glucuronide) in the sample by a method which is not a radioimmunoassay; c) comparing the concentration or concentrations with a urinary concentration or concentrations in a normal subject, so as to thereby diagnose hyperthyroism or hypothyroidism in the subject.

This invention provides modifications of the methods described herein, such as a method of determining whether a subject being treated with thyroxine is receiving a proper dosage of thyroxine which comprises: a) obtaining a suitable urine sample from the subject; b) comparing the concentration of any one or any combination of one or more of the compounds described herein (such as TSH, T3, T4, T3-sulfate and T4-glucuronide) in the sample by a method which is not a radioimmunoassay; c) comparing the concentration or concentrations with the range of urinary concentrations or concentrations for a normal subject, wherein if the concentration is not within the range determined for a normal subject, it indicates that the subject is not receiving the proper dosage of thyroxine. This invention provides a method of diagnosing a thyroid condition in a subject which comprises: (1) obtaining a urine sample from the subject; (2) modifying the urine sample such the concentration of any one or any combination of the compounds described herein (such as TSH, T3, T4, T3-sulfate and T4-glucuronide) can be determined; (3) comparing the concentration or concentrations with the range of urinary concentrations or concentrations for a normal subject, so as to thereby diagnose hyperthyroism or hypothyroidism in the subject. This invention provides a method of determining whether a subject being treated with thyroxine is receiving a proper dosage of thyroxine which comprises: (1) obtaining a urine sample from the subject; (2) modifying the urine sample such the concentration of any one or any combination of the compounds described herein (such as TSH, T3, T4, T3-sulfate and T4-glucuronide) can be determined; 3) comparing the concentration or concentrations with the range of urinary concentrations or concentrations for a normal subject, wherein if the concentration is not within the range determined for a normal subject, it indicates that the subject is not receiving the proper dosage of thyroxine.

As used herein, the urine may be modified in such a way so as to enable to determination of the concentrations being measured. In one embodiment, the urine is modified such that the concentrations being measured may be determined in the Abbott Laboratories Imx® System (Abbott Park, Ill.). In one embodiment, the urine sample is modified such that its pH is adjusted. In one embodiment, the pH may be adjusted such that it is within a range of 7.2 to 7.6. In another embodiment, the pH is adjusted such that it is within a range of 7.3 to 7.5. In another embodiment, the pH is adjusted to 7.43 with 1N NaOH, and then the urine sample is diluted with equal volumes of 0.01 M borate buffer, pH 7.43.

This invention provides a method of monitoring a subject being treated with thyroxine and ensuring that the subject receives the proper dosage of thyroxine which comprises: (1) determining whether the subject is receiving the proper dosage of thyroxine by one of the methods described herein; (2) adjusting the dosage if it is determined that the subject is not receiving the proper dosage; (3) repeating steps (1) through (2) throughout the course of the treatment, thereby monitoring the subject being treated with thyroxine and ensuring that the subject receives the proper dosage of thyroxine.

In one embodiment of the methods described herein, the agent of step (1) is immobilized. One skilled in the art would know various means for immobilizing an agent. For example, the agent may be immobilized on a solid support. In one embodiment, the agent is immobilized on a gold particle. In another embodiment, the agent is immobilized on a latex particle. In another embodiment, the agent is immobilized on a magnetic particle. In one embodiment, the solid support is a microtiter plate well. In another embodiment, the solid support is a bead. In a further embodiment, the solid support is a surface plasmon resonance sensor chip. The surface plasmon resonance sensor chip can have pre-immobilized streptavidin. In one embodiment, the surface plasmon resonance sensor chip is a BIAcore™ chip.

In one embodiment of the methods described herein, the urine sample is not concentrated. In another embodiment, the urine sample is concentrated so as to increase the concentration of the various components in the urine, thereby facilitating measurement. In one embodiment, the urine is concentrated by a process which includes centrifugation, precipitation, filtration (such as through a membrane or chromatographic medium, magnetic particle or electrophoresis).

In one embodiment of the methods described herein, the detectable marker is a colorimetric marker, a luminescent marker, or a fluorescent marker. One skilled in the art would know various other detectable markers.

In one embodiment of the methods described herein, the thyroid conditions is one which is not related to a pituitary effect. In one embodiment, the thyroid conditions is related to an abnormality of the thyroid gland.

In one embodiment of the methods described herein, the urine sample is modified such that its pH is adjusted to 7.43 with 1N NaOH, and then the urine sample is diluted with equal volumes of 0.01 M borate buffer, pH 7.43.

The invention described herein may be adapted such that the concentrations are determined by any means known to one skilled in the art. Such means include but are not limited to fluresence, polarized fluorescence, turbidity, chemiluminescence, agglutination, and methods of antigen-antibody reaction.

The agent which binds the compound to be measured (which may sometimes be referred to as a capture agent) may be any agent known by one skilled in the art to bind the compound. These include not only the embodiments described herein, such as antibodies or receptors, but also compounds with affinity, such as a lectin or protein A.

One skilled in the art would be able to determine agents which bind to the compounds being measured. Various agents are not only known to one skilled in the art, but are also publicly available. These agents may be determined by referring to an available source such as Linscott's Directory of Immunological and Biological Reagents (815 Whitney Way, Petaluma Calif. 94954; www.linscottsdirectory.com).

This invention will be better understood from the Experimental Details that follow. However, one skilled in the art will readily appreciate that the specific methods and results discussed are merely illustrative of the invention as described more fully in the claims that follow thereafter.

Supporting Data

Studies

Two series of over 100 patients in each group were studies in replication. In the first study, only TSH was measured. In the second study, in addition to TSH, triiodothyronine was measured in every patient's urine and thyroxine, thyroxine glucuronide and triiodothyronine were studied in selected cases.

Collection, Preparation and Storage of Urine Samples

For routine analysis, urine specimens were collected at the Boston Medical Center at approximately the same time as blood was drawn for TSH and other analyses. No “research” analyses were done on the sera-all serum tests were ordered for patient care. Urine specimens were transported by courier to the BioDx laboratory. In the first study, no urines were discarded except those estimated by the courier to be less than 10 ml in volume.

In the laboratory, those urines were centrifuged at 1,000 g for 10 min to remove particulate matter and volume, pH and specific gravity determined. For storage, a proprietary mixture containing buffer and preservatives (BioDx buffer) was added. This solution was shown not to interfere with determinations. Specimens from both studies were stored similarly at −20 degrees Centigrade.

The second study differed in certain respects. Specific gravity, pH albumin and creatinine were measured on each specimen by the Clinitek 50 Instrument, manufactured by Bayer Corporation and approved by the FDA for clinical analysis. In addition, total thyroxine levels were measured on some specimens by enzyme immunoassay (EIA) on microtiter plates obtained from BioTecx Diagnostics, Inc., Houston, Tex. Thyroxine measurements were not obtained on every specimen because the method could not be validated to our satisfaction. Total triiodothyronine (TT3) was measured on every specimen by the IMx procedure.

Validation of TSH Methodology

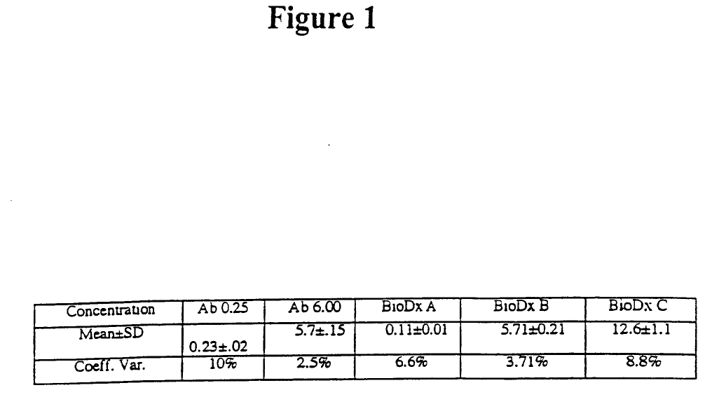

The TSH method was validated by conventional criteria as described above. The background readings for our standards were 0.01 μU/ml or less. The sensitivity of the Abbott method on serum is claimed to be 0.03 μU/ml. Our sensitivity was somewhat greater at about 0.02 μU/ml but, in analyzing the data, we accepted 0.05 μU/ml or less to be low. The intra-assay precision test on 10 aliquots of the same sample containing 0.05 μU/ml (done in duplicate) was 0.05±/−0.0, obviously a coincidence. With concentrations of 5 μU/ml, the mean measurement was 5.25 with S.D. of 0.15. The intra-assay coefficient of variability was 2.83%. The inter-assay precision was determined over a period of six months (FIG. 1): Abbott's standards of 0.25 μIU/ml and 6 μIU/ml are serum standards. BioDx standards in the first study were established from a patient pool containing uncertain amounts. The pool was filtered through Whatman no. 1 filter paper. Standards A, B and C represent about 0.1, 6 and 12 μU/ml respectively determined by repeated measurement and appropriate dilutions.

The BioDx urinary standards have precision coefficients similar to those reported by Abbott for their serum standards. In the second study, a standard pool was established by collecting urine from two volunteers who were judged to be normal by blood measurements of TSH and thyroid hormones. Ten liters were collected, filtered through 0.22 micron Falcon filter to remove bacteria and certain hydrophobic substances including most of the TSH, T3 and T4. The pool was divided into a series of sub-pools and appropriate amounts of TSH, T3 and T4 added to prepare standards of a variety of concentrations. These sub-pools were aliquoted into 2 ml vials and stored as above. TSH, T4 and T3 from these pools were measured monthly and, in confirmation of the first study, concentrations were unchanged for over a year.

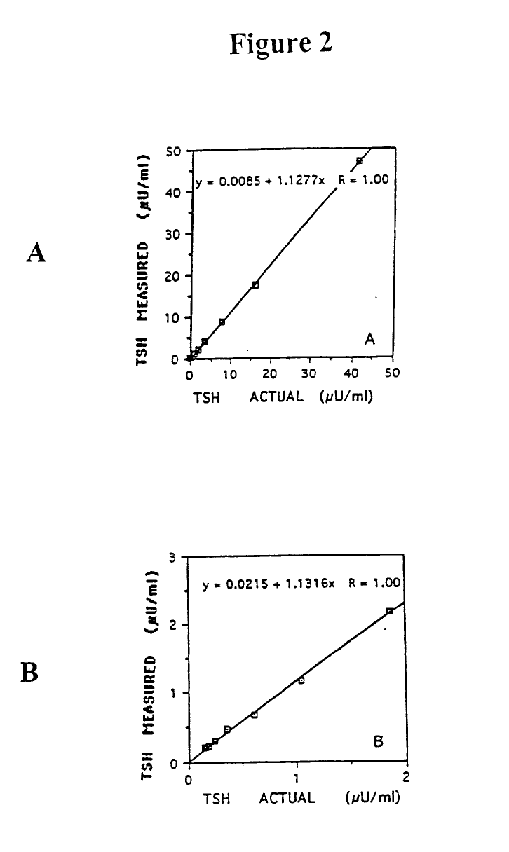

The accuracy of the method was determined by adding 41 μU human TSH to urine containing 0.13 μU and then serially diluting that solution down to 0.04 μU/ml (FIGS. 1A and 1B). FIGS. 2A and 2B differ in the range of concentrations presented. The correlations are excellent. Replicate experiments with different urines had correlation coefficients over 0.9. Nevertheless, in stability experiments in which 10 replicates were re-measured over many days, on rare occasions, a single aberrant value (out of 10) was obtained. It was clearly attributable to the automation.

In summary, the adaptation of the IMx to urinary TSH measurements is as sensitive, accurate and reproducible as it is for serum.

Stability of TSH Standards and Specimens

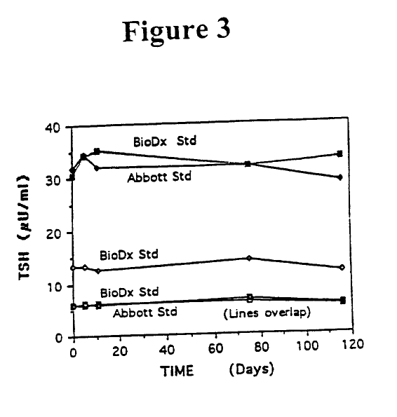

The stability of TSH in urine and serum was determined at room temperature, 4 C. and −20 C. BioDx stabalizing solution was added to each sample (FIG. 3). At room temperature and refrigerator temperature, TSH was stable for over a year. In fact, more differences were attributable to the instability of the instrument than the samples. After 8 months, some urine specimens, but not standards, showed loss of activity, especially if not stored in BioDx buffer. However, those urine specimens that were passed through 0.22 micron filter were stable for over a year which is the longest that we have been able to test them.

Factors Affecting the Assay of TSH

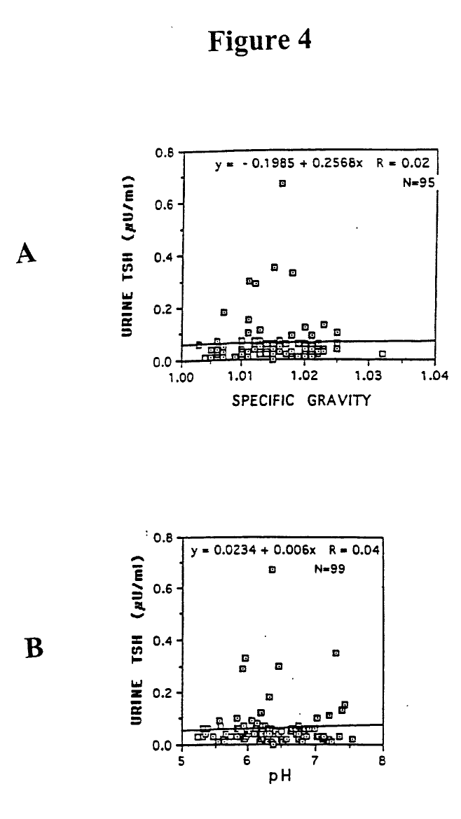

The effects of specific gravity and pH were examined. Of the 100 consecutive samples (with no exclusions other than volumes under 10 ml and missing data), no effect of either pH or specific activity could be demonstrated (FIGS. 4A and B).

In the second study, we also examined the effects of albumin and creatinine concentrations on the TSH measurements. The data were not influenced by concentrations of these substances within the ranges in patients with normal renal function.

Clinical TSH Findings

In the first study, urines and bloods were collectd from 102 patients at the Boston Medical Center Endocrinology Clinic. TSH was determined at the hospital laboratory by chemiluminescence on a Bayer instrument. The only modification to routine patient diagnosis and care were the urine collections. We(and others) have compared the results of Abbott's nephelometry method with Bayer's chemiluminescence method and found them comparable although causes of error may be totally different. (Enhancement and quenching of chemiluminescence as compared to interference by alien particles affecting nephelometry.) Because of specimen or data losses, some tables and figures presented below may have numerical differences of 2 or 3 patients.

The findings of the second study essentially replicated the first study except that it was more detailed in terms of characterization of the patients and had more determinations. The patients were initially divided into three categories: those who were considered to be normal, those taking thyroxine medication, and those taking tapazole medication. Various states of thyroid function occurred in each of the categories. We could not demonstrate significant differences among the three categories as to normal range, significance of high levels or significance of low levels.

Demonstration of a Discriminant Concentration of TSH in Urine of Patients

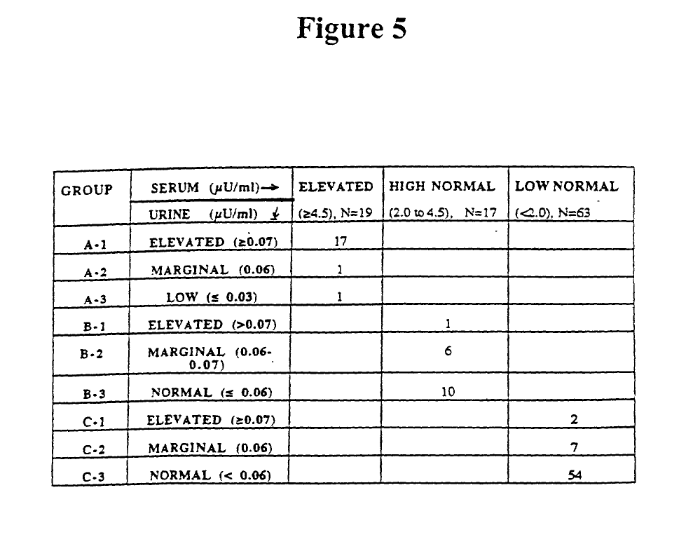

In the first study of over 100 patients, we designated discriminant concentrations that would establish the range of normal values. Serum TSH levels were ranked by concentration and cut-off levels established between 0.03 μU/ml and 0.07 μU/ml. The urinary TSH levels were then sorted according to FIG. 5. In the second study, the discriminant concentrations were about 25% higher. The reason is that levels of TSH in filtered urine, as measured in the first study, is about 25% less than in non-filtered urine. Furthermore, these numbers are based upon our standard of recombinant TSH which, when converted to WHO Reference preparation Units, increases again by about 20%.

Interpretation of Data

There are many complex sources of error in these data. The two different kinds of commercial instruments and even the same instruments on different body fluids may be in disagreement. For example, in the first series, in Groups B-1, B-2 and C-2, it is extremely unlikely that low serum levels of TSH would be accompanied by inappropriately high urine levels. Most likely, one or the other method is wrong and, we believe that the probability is equal for either source. One patient in the A group had a urinary TSH (0.03 U/ml) associated with the increased serum TSH (4.52 U/ml). False negatives are of more concern than false positives that would be followed up by a physician. Three prior serum determinations on this patient were less than 3 while she was being maintained on 88 μg of thyroxine. It probably indicates that the serum value was aberrant. In the marginal group, about 0.06 is the greatest source of error. Certainly some of this error is methodology but a more significant and interesting consideration is that all but four of these patients were being treated with thyroxine. Because of the clinical setting, we did not know the interval of time between thyroxine ingestion and blood and urine collection. The others were untreated or on tapazole. In summary, in this series as in many such series, lower levels of the high range overlap higher levels of the normal range. In the lowest range, two reports were inappropriate. This must be technical error because urinary TSH cannot appear from blood levels that are one-third the urinary concentration. We corrected some of these errors in the second study. We were able to identify certain drugs that might have affected the urinary levels. For example, Premarin® itself may affect levels both biologically by increasing thyroxine binding proteins and instrumentally by affecting the detection. Also, IMx directions state that acetylsalicyclic acid may affect the measurements; many patients take aspirin. Nevertheless, our data on blood determinations as well as the published data on blood determinations of TSH show variations that exceed the urinary concentrations. Even the accepted normal range of 0.03 μIU/ml to about 5.5 μIU/ml is almost a 20-fold range whereas the urinary range is about 10-fold.

Special conditions-Thyroxine and Pregnancy

In the first study, 35 patients were monitored by both serum and urine analyses while being treated with thyroxine for a variety of clinical conditions. They received doses of thyroxine between 50 to 150 μg/day. Only two patients did not conform to the limits of 4.5 μU/ml for serum and 0.07 μU/ml for urine. Similar data were obtained in the second series although, in that series, control concentrations were not as good as in the first series. Urinary measurements were as useful as serum measurements to detect proper medication although in a few patients there was disagreement between the two indicators.

Serum levels of TSH were determined weekly for as long as a year in eight breast-feeding women to determine if elevations occurred as a result of breast-feeding. The range of values for 122 determinations was 0.37 to 3.71 with standard deviations for each patient less that 10%. Eleven urine levels completed in one patient whose serum TSH ranged from 1.25 to 2.1 were uniformly at the lower limit of normal.

Studies On Thyroxine and Triiodothyronine

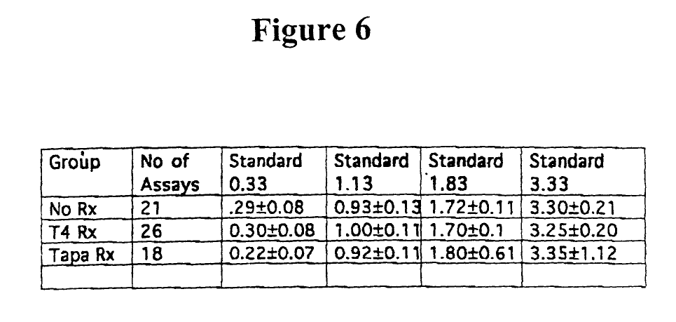

Establishment of Standards: The urine used for the second group of TSH patients described above was used to establish standards of the thyroid hormones. It was not possible to establish standards of the glucuronic acid and sulfuric acid conjugates because, insofar as we know, they have never been synthesized prior to this study. Thyroxine and triiodothyronine at high, medium and low concentrations expected to be found in urine were aliquotted into small vials similarly to the TSH allotments, described above. By adding and then measuring various amounts of T3 to the T3-depleted urine standard, we established the accuracy of the IMx Total triiodothyronine test for urine. We established the intra-assay precision by measuring 10 samples of standards containing 1.0 ng/ml repetitively in a single assay. The samples were placed at different positions in the instrument's carousel to test for errors that might arise from position. The mean of 10 determinations was 1.11 with a standard deviation (SD) of 0.01. Thus the intra-assay precision of the urinary standards was similar to that cited by the IMx pamphlet for serum. The inter-assay precision was validated by comparing the same standards over the course of routine assays of the untreated group (21 assays), the thyroxine treated group (26 assays) and the group treated with tapazole (18 assays). The standards tested contained 0.33, 1.13, 1.83 and 3.33 ng/ml. The mean recoveries are presented in FIG. 6. Similar results were obtained for the Abbott standards in their medium although the Abbott kit does not contain a standard near 0.3, their low limit of sensitivity. The respective coefficients of variation are 0.12, 0.09 and 0.05 for the Abbott standards and 0.12, 0.15 and 0.18 for the urine standards, the last number reflecting one poor series. However, Abbott does not offer a standard 0.03 μIU/ml while our standard's coefficient was 0.28. This obviously is at the very limit of sensitivity and shows unreliability below that point. As mentioned above, ordinarily, we do not rely upon any determination less than 0.05.

From these data, it is also clear that the measurement curve is linear and that the extinction point is about 0.3 ng/ml. Stability of urinary T3 was tested for time and temperature. The urines were stable for months in the frozen state and for over 3 days at room temperature provided that they were stored with at least 20% (v/v) of buffer. In the native state, however, the urinary T3 deteriorated in both environments at an accelerated rate. The T3 standards were stable under all conditions.

Measurement of Thyroxine

After studying many methods, the method selected for the mesurement of thyroxine was the enzyme-linked immunoassay kit sold by BioTex, Houston, Tex. Data collected by this method were erratic. Nevertheless, by performing many assays on several dilutions of the same urines, we were able to establish that, in general, thyroxine levels equaled triiodthyronine levels.

Although the IMx method was not sensitive enough to measure total thyroxine reliably, it did measure free T4 satisfactorily. Triiodothyronine was measured reliably in the IMx Total T3 system as described above. However, this procedure was very sensitive to interference especially from certain drugs. For example, we extracted some Premarin® capsules and demonstrated that the dye on the surface of the 0.625 ng tablet gave very high readings. Premarin, itself may contain interfering substances.

We identified some false positives by levels were at least ten times higher than occurred in our “high” samples and confirmed the artifact by destroying T3 in the sample and demonstrating that the high levels remained intact.

Measurement of Glucuronidated T4 and T3

Thyroxine glucuronidate in urine was hydrolysed with Sigma bacterial β-glucuronidase. The time interval for hydrolysis was tested from 1 to 24 hours under the conditions recommended by Sigma. It was also hydrolyzed by 0.2 N hydrochloric acid at pH 2 over a period of less than one hour. However, in spite of examination of many samples of urine hydrolyzed over extended periods of time, we found no evidence of triiodothyronine glucuronide.

Acid hydrolysis needs to be done cautiously because the acid can de-iodinate thyroxine converting T4 to T3 or RT3 and possible other substances [13]. Furthermore, we found considerable interference from the acid, even when buffered, in the IMx T3 test. Thus, elevated salt concentrations affected the assay.

Measurement of sulfated T4 and T3

We found that certain animal sulfatases interferes with the IMx method. Bacterial sulfatase (Sigma) was used as directed. We found little evidence of the presence of thyroxine sulfate in urine in our preliminary studies but significant levels of triiodothyronine-sulfate were present. To confirm our data, triodothyronine sulfate was synthesized by methods of Levitz (personal communication) for radioactive estrone glucuronide. T3-sulfate gave a unique spectrum with a major absorption maximum at 362 nm as compared to triiodothyronine whose major absorption maxima occurred at 318. Hydrolysis of the synthetic T3-sulfate with sulfatase resulted in the original T# as tested in the Imx system. Thyroxine-sulfate did not interfere in the measurement of T3. Nevertheless, the complex was unstable and rapidly deteriorated as was the case with the natural sulfate when not stored in liquid form in appropriate buffer.

Concentrations of T3-sulfate were determined by measuring Total T3 in the samples before and after hydrolysis with the sulfatase and subtracting the initial concentration. The levels of T3-sulfate varied from one-third to one-half that of total T3.

In summary, we found little thyroxine sulfate and little triiodothyronine glucuronide. The concentrations of T4-glucuronide and T3-sulfate were about one-third to one-half of the parent compound.

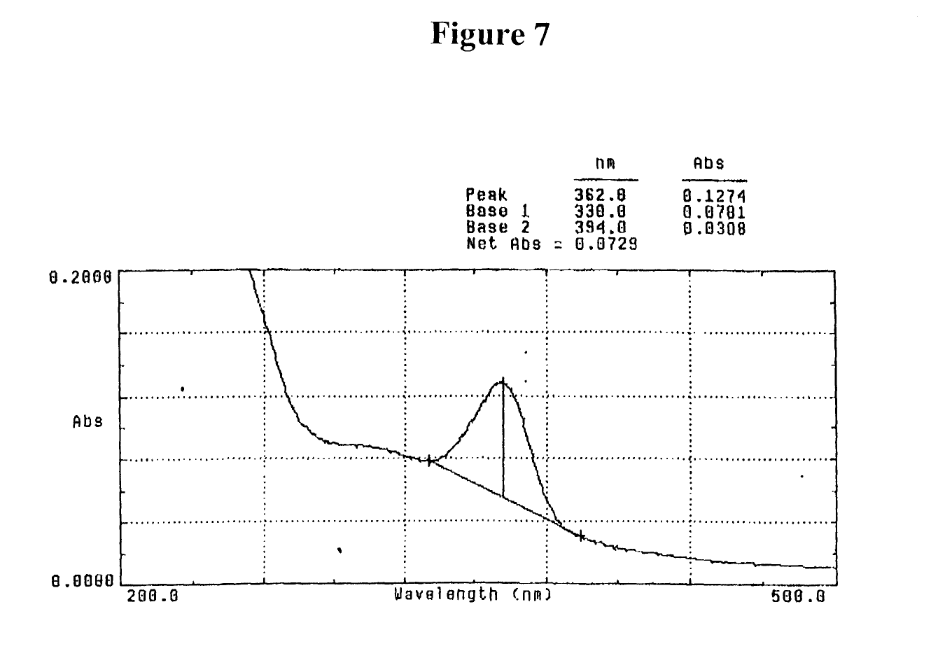

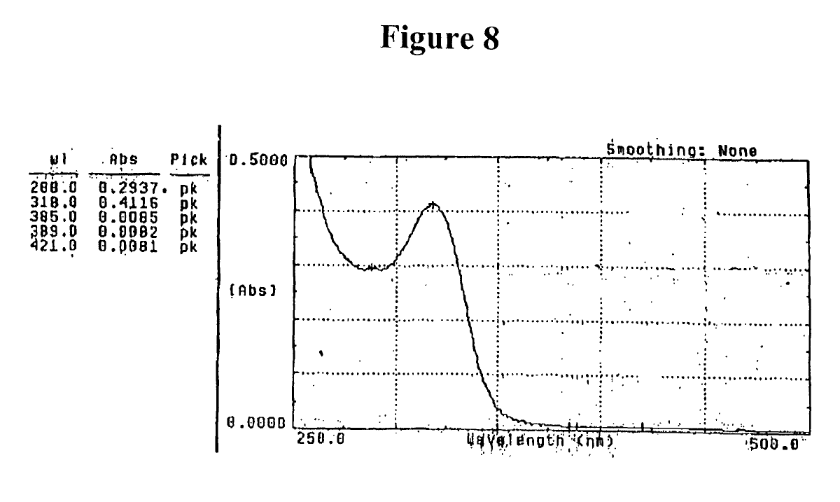

Figures of spectra of T3 (FIG. 7) and T3-sulfate (FIG. 8).

Concentrations of T3 in Patients Urine

We compared T3 concentration in three groups: those not taking any thyroid medication, those taking thyroxine daily and those taking an inhibitor of thyroxine secretion, either tapazole or propylthiouracil. No differences could be detected among the three groups in consideration of their actual thyroid state. The overall mean derived from 92 patients was 0.97 ng/ml with a range of 0.09 and a maximum of 3.685. The median was 0.8. As measured in relation to the standards cited above which were generally somewhat lower than the actual content, these levels might also be somewhat lower than the actual concentrations. Review of the data indicates that the lower limit that defines a hypothyroid state would be 0.3 ng/ml or less. The upper limit might be 1.5 ng/ml or more.

Effects of Thyroxine Therapy

The importance of monitoring thyroxine therapy necessitated analysis of the parameter that might influence results in patients taking that medication. In over 30 patients, we were able to identify the interval of time between the ingestion of the medication and the collection of the urine and blood specimens. They were taking doses of 50 to 150 mg/day with the majority consuming 100+/−25 mg/day.

The levels that we measured for both TSH and T3 were not grossly different in the group of patients that received thyroxine therapy than in those getting suppressive therapy (e.g. tapazole) or not getting any therapy. The interval between ingestion of medication and testing appeared to have an effect after 24 hours but these data need to be extended. Thus, the use of TSH and T3 to monitor therapy is clearly valid.

Effects of Replication

Replication is a powerful test of veracity. It is not cost-effective to ask a patient to perform three or four tests to verify thyroid status at >$50/test on blood but it is possible to replicate diagnosis and therapy assessment by a routine method such as a home test. Without defining the intervals that would be requested by the physician, we can emphasize that statistically the power of replication is enormous in clinical testing. For example, if the blood test were accurate about 75% of the time and the urine test were accurate only 50% of the time, two or three urine tests would have greater validity than the sole blood.

Kit Development

The TSH or thyroid hormone or thyroid metabolite kit may be developed in various ways. There are three approaches to the problem of low concentrations.

Traditional tests such as the lateral flow devices (e.g. dipsticks) can be made to be more sensitive by enhancing the read-outs. One example of that method would be to use the gold particle in a way that silver aggregates would enhance the reading without introducing instrumentation.

Another method would be to use an instrument that might measure a different physical parameter than visual light. An example of that would be an instrument that might mobilize fluorescence.

Another method would be to concentrate the TSH or other antigen to the sensitivity of the test. Examples of that method would be the use of magnetic particles, centrifugation (for the physician's office) or filtration through a retentive membrane.

Many of these techniques are in common use. The model of human chorionic gonadotropin tests (pregnancy tests) is that beta sub-unit particle captures the antigen (e.g. TSH) and the chromophoric detection system utilizes a site on the alpha sub-unit.

Tests are only as good as the reagents. Excellent TSH antigen is available from Seradyne, Indianapolis, IN which bought it from Genzyme. High affinity monoclonal antibodies or polyclonal antibodies that were affinity-purified are available from many sources (e.g. BioDesign, Fitzgerald, etc.).

The TSH kit may developed in various methods including but not limited to a lateral flow device (dipstick), or as a flow-through test. The lateral flow device is described above. In the flow-through test, the analyte is collected on a membrane by a capture antibody and then the detector agents are applied. For example, a beta-sub-unit antibody might capture the TSH and an alpha sub-unit would carry a chromophoric detector system. Both are in common use and many good development companies are able to make the test at a cost of less than a dollar. The caveats are using good reagents and adequate amounts of analyte. The TSH reagents are excellent being derived from recombinant TSH prepared by Genzyme and monoclonal antibodies of very high affinity. Good T4 and T3 antibodies are also available from many sources (e.g., BioDesign, Fitzgerald, etc.).

IMx System

The invention described herein may be performed in the IMx® system developed by Abbott Laboratories (Abbott Park, Ill.). Abbott describes such system as one for use in quantitating the substance (e.g. T3 or TSH) in human serum. Protocol for measuring substances in serum are described in the Abbott Laboratory brochures such as those having the following titles: (1) Imx® System for Total T3, published in January 1998 by Abbott Laboratories; and (2) Imx® System for Ultrasensitive hTSHII, published in August 1997 by Abbott Laboratories. It is important to stress than the brochures for the Imx system specify that the serum consentration is determined. In contrast, the present invention relates to the measurement of these analytes in urine. Accordingly, in order for the urine sample to be tested, it may be modified such that the urine pH is adjusted to 7.43 with 1N NaOH. The urine mau then be diluted with equal volumes of 0.01M borate buffer, pH 7.43.

Is an immunoassay which uses fluorogenic enzyme substrates and fluroescence polarization techniques. The procedure may use a coated submicron microparticle as the means by which the analyte to be measured is captured.

Description of Test

Kits for TSH, T3 and T4 may be made and they should be used simultaneously. One model for the TSH kit is the dipstick or cassette similar to those used to detect hCG in urine utilizing the principles of immunochromatography. In theory, the antigen, TSH, in urine will be filtered to remove cells and particles through a sample pad that accepts a certain amount, usually about 0.2 ml of urine. A conjugate pad containing reagents reacts the antigen with the capture antibody (anti-β sub-unit). It migrates along a nitrocellulose transport membrane until it reaches a line where the aggregate is captured by a fixed detector antibody (e.g., anti-α sub-unit) where it is read out. A sponge at the end of the membrane absorbs the fluid. There are numerous versions of this system. Popular detectors are colloidal gold or latex particles because they are more stable than enzymes, can have a long shelf life and are very sensitive. The read-out is usually positive or negative although semi-quantitation can be achieved by placing a series of lines. A similar pattern cannot be devised for smaller molecules such as T3 because a double antibody method for that molecule is not easily developed. A competitive assay or enzyme-linked immunoassay is conventionally used for such molecules.

The tests are rapid (2 to 5 minutes), simple to perform and interpret, and very convenient. In addition to working out details of reagents, especially antibodies and concentrations, a very important aspect is finding the appropriate discriminant level that separates positive from negative read-outs. Results are positive or negative; no quantitative cassette method has yet been developed. The proper discriminant level is determined from clinical data or comparison with an established test as we have done with the Abbott kit on the IMx instrument. Tests with different discriminant levels are not difficult to make if the quantity of analyte in the urine is sufficient.

References

1. Nicoloff, J. T. and C. A. Spencer, The use and misuse of the sensitive thyrotropin assays. J Clin Endocrinol Metab, 1990. 71: 553-558.

2. Kaptein, E. M. and J. C. Nelson, Serum thyroid hormones and thyroid stimulating hormone, in Atlas of Clinical Endocrinology: Thyroid Diseases, M. I. Surks, Ed. 1999, Current Medicine: Philadelphia. 15-31.

3. Chopra, I. J., Nature, sources and relative biologic significance of circulating thyroid hormones, in Werner and Ingbar's The Thyroid, L. E. Braverman & R. D. Utiger, Eds. 1991, J. B. Lippincott: Philadelphia.

4. Chan, V., et al., Urinary triiodothyronine excretion as an index of thyroid function. Lancet, 1972. ii (August 5): 253-256.

5. Burke, C. W., R. A. Shakespear, and T. R. Fraser, Measurement of thyroxine and triiodothyronine in human urine. Lancet, 1972(December 2): 1177-1179.

6. Patel, Y. C., F. P. Alford, and H. G. Burger, The 24-hour plasma thyrotropin profile. Clin Sci, 1972. 43: 71-77.

7. Stockigt, J. R., Serum Thyrotropin and Thyroid Hormone Assessments and Assessment of Thyroid Hormone Transport, in Werner and Ingbar's The Thyroid, L. E. Braverman and R. D. Utiger, Editors. 1991, J. B. Lippincott: Philadelphia.

8. Hertz, S. and E. G. Oastler, Assay of blood and urine for thyreoptropic hormone in thyrotoxicosis and myxedema. Endocrinology, 1936. 20: 520-525.

9. Kuku, S. F., et al, Concentrations of immunoreactive thyrotrophic hormone in urine of normal subjects, patients with thyroid disorders and hypopituitarism, and after infusion of human thyrotrophic hormone. J. Endocrin., 1974. 62: 645-655.

10. Kuku, S. F., et al., Sephadex gel filtration analysis of immunoreactive thyrotrophic hormone in human urine. J. Endocrin., 1974. 62: 657-662.

11. Van Herle, A. J., et al., Immunoreactive “TSH” in urinary concentrates of Graves' disease patients: a radioimmoassay artifact. Eur. J. Clin. Invest, 1978. 8: 295-301.

12. Hufner, M. and R. D. Hesch, Triiodothyronine determinations in urine. Lancet, 1973(January 13 (Letter to Editor)): 101-102.

13. Gaitan, J. E., et al, Measurement of triiodothyronine in unextracted urine. J Lab clin Med, 1975. 86: 538-546.

14. Yoshida, K., et al, Measurement of triiodothyronine in urine. Tohoku J exp Med, 1980. 132: 389-395.

15. Haddow, J. E., et al., Maternal thyroid deficiency during pregnancy and subsequent neuropsychological development of the child. N Engl J Med, 1999. 341: 549-555.

16. Hollowell, J. G., et al., Iodine nutrition in the United States. Trends and public health implications: Iodine excretion data from National health and Nutrition Examination Surveys I and III (1971-1974 and 1988-1994). J Clin Endocrinol Metab, 1988. 83: 3401-3408.

17. Amino, N., et al, Therapeutic Controvery: Screening for postpartum thyroiditis. J Clin Endocrinol Metab, 1999. 84: 1813-1821.

18. Ortiz, R., et al., Effect of early referral to an endocrinologist on efficiency and cost of evaluation and development of treatment plan in patients with thyroid nodules. J Clin Endocri Metab, 1998. 83:3803-3807.

19. Tunbridge, W. M. G., et al., The spectrum of thyroid disease in a community: the Whickham survey. Clin Endocrinol, 1997. 7: 481-493.

20. Sawin, C. T., et al., The aging thyroid: thyroid deficiency in the Framingham Study. Arch Intern Med, 1985. 145: 1386-1388.

21. Sawin, C. T., Thyroid dysfunction in older persons. Adv Intern Med, 1991. 37: 223-248.

22. Danese, M. D., et al., Screening for mild thyroid failure at periodic health examination, a decision and cost-effectiveness analysis. JAMA, 1996. 276: 285-292.

23. Singer, P., et al., Treatment guidelines for patients with hyperthyroidism and hypothyroidism. JAMA, 1995. 273: 808-812.

24. Scanlon, M. F. and A. D. Toft, Regulation of thyroid secretion, in Werner and Ingbar's The Thyroid, L. E. Braverman and R. D. Utiger, Editors. 1996, J. B. Lippincott: Philadelphia. p. 220-240.

25. Tanis, B. C., R. G. J. Westendorp, and A. Smelt, H. M., Effect of thyroid substitution on hypercholesterolemia in patients with sub-clinical hypothyroidism: a reanalysis of intervention studies. Clin Endocrinol, 1996. 44: 643-649.

What is claimed:

1. A method of diagnosing a thyroid condition in a subject which comprises:
a) obtaining a suitable urine sample from the subject;
b) determining the concentration of thyroid stimulating hormone in the sample by a method which is not a radioimmunoassay; and
c) comparing the concentration of thyroid stimulating hormone with a urinary concentration of thyroid stimulating hormone in a normal subject; wherein:
i) a concentration of thyroid stimulating hormone which is higher than the urinary concentration of thyroid stimulating hormone in the normal subject diagnoses hypothyroidism in the subject; and
ii) a concentration of thyroid stimulating hormone which is lower than the urinary concentration of thyroid stimulating hormone in the normal subject diagnoses hyperthyroidism in the subject.

2. The method of claim 1, wherein step (b) comprises:
(1) contacting an agent capable of binding to thyroid stimulating hormone with the urine sample so as to bind thyroid stimulating hormone which is present in the sample to the agent;
(2) removing unbound urine sample;
(3) contacting the bound thyroid stimulating hormone with a detectable agent capable of binding to thyroid stimulating hormone so as to bind the detectable agent to the bound thyroid stimulating hormone;
(4) removing unbound detectable agent; and
(5) determining the amount of detectable agent which is bound to the thyroid stimulating hormone, thereby determining the amount of thyroid stimulating hormone in the urine sample.

3. The method of claim 2, wherein the agent capable of binding to thyroid stimulating hormone of step (1) is an antibody which binds to thyroid stimulating hormone.

4. The method of claim 2, wherein the agent capable of binding to thyroid stimulating hormone of step (1) is a thyroid stimulating hormone receptor.

5. The method of claim 2, wherein the detectable agent is an antibody which binds to an epitope on thyroid stimulating hormone which differs from the epitope to which the agent of step (1) binds.

6. The method of claim 2, wherein the detectable agent is labeled with a detectable marker.

7. The method of claim 1, wherein a concentration greater than 0.35 μIU/ml diagnoses hypothyroidism in the subject.

8. The method of claim 1, wherein a concentration less than 0.04 μIU/ml diagnoses hyperthyroidism in the subject;

9. A method of diagnosing a thyroid condition in a subject which comprises:
a) obtaining a suitable urine sample from the subject;
b) determining the concentration of triiodothyronine in the sample by a method which is not a radioimmunoassay; and
c) comparing the concentration of triiodothyronine with a urinary concentration of triiodothyronine in a normal subject; wherein
i) a concentration of triiodothyronine which is lower than the urinary concentration of triiodothyronine in the normal subject diagnoses hypothyroidism in the subject; and
ii) a concentration of triiodothyronine which is higher than the urinary concentration of triiodothyronine in the normal subject diagnoses hyperthyroidism in the subject.

10. The method of claim 9, wherein step (b) comprises:
(1) contacting an agent capable of binding to triiodothyronine with a pre-determined amount of detectable triiodothyronine and the urine sample, so as to form a complex between the agent and (i) the detectable triiodothyronine or (ii) the triiodothyronine present in the urine sample;
(2) determining the amount of detectable triiodothyronine which is bound to the agent, wherein the difference between the predetermined amount of detectable triiodothyronine and the amount of detectable triiodothyronine which is bound indicates the amount of triiodothyronine present in the urine sample.

11. The method of claim 9, wherein step (b) comprises:
(1) contacting an agent capable of binding to triiodothyronine with a pre-determined amount of detectable triiodothyronine and the urine sample, so as to form a complex between the agent and (i) the detectable triiodothyronine or (ii) the triiodothyronine present in the urine sample;
(2) determining the amount of detectable triiodothyronine which is not bound to the agent, thereby determining the amount of triiodothyronine present in the urine sample.

12. The method of claim 10 or 11, wherein the agent of step (1) which is capable of binding to triiodothyronine is an antibody.

13. The method of claim 10 or 11, wherein the agent of step (1) which is capable of binding to triiodothyronine is a triiodothyronine receptor.

14. The method of claim 10 or 11, wherein the detectable triiodothyronine is labeled with a detectable marker.

15. The method of claim 9, wherein a concentration less than 0.3 ng/ml diagnoses hypothyroidism in the subject.

16. The method of claim 9, wherein a concentration greater than 1.5 ng/ml diagnoses hyperthyroidism in the subject.

17. A method of diagnosing a thyroid condition in a subject which comprises:
a) obtaining a suitable urine sample from the subject;
b) determining the concentration of triiodothyronine-sulfate in the sample by a method which is not a radioimmunoassay; and
c) comparing the concentration of triiodothyronine-sulfate with a urinary concentration of triiodothyronine-sulfate in a normal subject; wherein
i) a concentration of triiodothyronine-sulfate which is lower than the urinary concentration of triiodothyronine-sulfate in the normal subject diagnoses hypothyroidism in the subject; and
ii) a concentration of triiodothyronine-sulfate which is higher than the urinary concentration of triiodothyronine-sulfate in the normal subject diagnoses hyperthyroidism in the subject.

18. The method of claim 17, wherein step (b) comprises:
(1) contacting an agent capable of binding to triiodothyronine-sulfate with a pre-determined amount of detectable triiodothyronine-sulfate and the urine sample, so as to form a complex between the agent and (i) the detectable triiodothyronine-sulfate or (ii) the triiodothyronine-sulfate present in the urine sample;
(2) determining the amount of detectable triiodothyronine-sulfate which is bound to the agent, wherein the difference between the pre-determined amount of detectable triiodothyronine-sulfate and the amount of detectable triiodothyronine-sulfate which is bound indicates the amount of triiodothyronine-sulfate present in the urine sample.

19. The method of claim 18, wherein step (b) comprises:
(1) contacting an agent capable of binding to triiodothyronine-sulfate with a pre-determined amount of detectable triiodothyronine-sulfate and the urine sample, so as to form a complex between the agent and (i) the detectable triiodothyronine-sulfate or (ii) the triiodothyronine-sulfate present in the urine sample;
(2) determining the amount of detectable triiodothyronine-sulfate which is not bound to the agent, thereby determining the amount of triiodothyronine-sulfate present in the urine sample.

20. The method of claim 18 or 19, wherein the agent of step (1) which is capable of binding to triiodothyronine-sulfate is an antibody.

21. The method of claim 18 or 19, wherein the agent of step (1) which is capable of binding to triiodothyronine-sulfate is a triiodothyronine receptor.

22. The method of claim 18 or 19, wherein the detectable triiodothyronine-sulfate is labeled with a detectable marker.

23. The method of claim 17, wherein a concentration lower than 0.1 ng/ml diagnoses hypothyroidism in the subject.

24. The method of claim 17, wherein a concentration higher than 0.5 ng/ml diagnoses hyperthyroidism in the subject.

25. A method of diagnosing a thyroid condition in a subject which comprises:
a) obtaining a suitable urine sample from the subject;
b) determining the concentration of thyroxine present in the sample by a method which is not a radioimmunoassay;
c) comparing the concentration of thyroxine with a urinary concentration of thyroxine in a normal subject; wherein
i) a concentration of thyroxine which is lower than the concentration of thyroxine in the normal subject diagnoses hypothyroidism in the subject; and
ii) a concentration of thyroxine which is higher than the urinary concentration of thyroxine in the normal subject diagnoses hyperthyroidism in the subject.

26. The method of claim 25, wherein step (b) comprises:
(1) contacting an agent capable of binding to thyroxine with a pre-determined amount of detectable thyroxine and the urine sample, so as to form a complex between the agent and (i) the detectable thyroxine or (ii) the thyroxine present in the urine sample;
(2) determining the amount of detectable thyroxine which is bound to the agent, wherein the difference between the pre-determined amount of detectable thyroxine and the amount of detectable thyroxine which is bound indicates the amount of thyroxine present in the urine sample.

27. The method of claim 25, wherein step (b) comprises:
(1) contacting an agent capable of binding to thyroxine with a pre-determined amount of detectable thyroxine and the urine sample, so as to form a complex between the agent and (i) the detectable thyroxine or (ii) the thyroxine present in the urine sample;
(2) determining the amount of detectable thyroxine which is not bound to the agent, thereby determining the amount of thyroxine present in the urine sample.

28. The method of claim 26 or 27, wherein the agent of step (1) which is capable of binding to thyroxine is an antibody.

29. The method of claim 26 or 27, wherein the agent of step (1) which is capable of binding to thyroxine is a thyroxine receptor.

30. The method of claim 26 or 27, wherein the detectable thyroxine is labeled with a detectable marker.

31. The method of claim 25, wherein a concentration lower than 0.3 ng/ml diagnoses hypothyroidism in the subject.

32. The method of claim 25, wherein a concentration higher than 1.5 ng/ml diagnoses hyperthyroidism in the subject.

33. A method of diagnosing a thyroid condition in a subject which comprises:
a) obtaining a suitable urine sample from the subject;
b) determining the concentration of thyroxine-glucuronide present in the sample by a method which is not a radioimmunoassay;
c) comparing the concentration of thyroxine-glucuronide with a urinary concentration of thyroxine-glucuronide in a normal subject; wherein
i) a concentration of thyroxine-glucuronide which is lower than the concentration of thyroxine-glucuronide in the normal subject diagnoses hypothyroidism in the subject; and
ii) a concentration of thyroxine-glucuronide which is higher than the urinary concentration of thyroxine-glucuronide in the normal subject diagnoses hyperthyroidism in the subject.

34. The method of claim 33, wherein step (b) comprises:
(1) contacting an agent capable of binding to thyroxine-glucuronide with a pre-determined amount of detectable thyroxine-glucuronide and the urine sample, so as to form a complex between the agent and (i) the detectable thyroxine-glucuronide or (ii) the thyroxine-glucuronide present in the urine sample;
(2) determining the amount of detectable thyroxine-glucuronide which is bound to the agent, wherein the difference between the predetermined amount of detectable thyroxine-glucuronide and the amount of detectable thyroxine-glucuronide which is bound indicates the amount of thyroxine-glucuronide present in the urine sample.

35. The method of claim 33, wherein step (b) comprises:
(1) contacting an agent capable of binding to thyroxine-glucuronide with a pre-determined amount of detectable thyroxine-glucuronide and the urine sample, so as to form a complex between the agent and (i) the detectable thyroxine-glucuronide or (ii) the thyroxine-glucuronide present in the urine sample;
(2) determining the amount of detectable thyroxine-glucuronide which is not bound to the agent, thereby determining the amount of thyroxine-glucuronide present in the urine sample.

36. The method of claim 34 or 35, wherein the agent of step (1) which is capable of binding to thyroxine-glucuronide is an antibody.

37. The method of claim 34 or 35, wherein the agent of step (1) which is capable of binding to thyroxine-glucuronide is a thyroxine receptor.

38. The method of claim 34 or 35, wherein the detectable thyroxine-glucuronide is labeled with a detectable marker.

39. The method of claim 33, wherein a concentration lower than 0.1 ng/ml diagnoses hypothyroidism in the subject.

40. The method of claim 33, wherein a concentration higher than 0.5 ng/ml diagnoses hyperthyroidism in the subject.

41. A method of diagnosing a thyroid condition in a subject which comprises:
(2) obtaining a suitable urine sample from the subject;
b) determining the concentration of thyroid stimulating hormone and the concentration of triiodothyronine in the sample by a method which is not a radioimmunoassay;
c) comparing the concentration of thyroid stimulating hormone with a urinary concentration of thyroid stimulating hormone in a normal subject and comparing the concentration of triiodothyronine with a urinary concentration of triiodothyronine in a normal subject; wherein
i) a concentration of thyroid stimulating hormone which is higher than the urinary concentration of thyroid stimulating hormone in the normal subject, and a concentration of triiodothyronine which is lower than the urinary concentration of triiodothyronine in the normal subject, diagnoses hypothyroidism in the subject; and
ii) a concentration of thyroid stimulating hormone which is lower than the urinary concentration of thyroid stimulating hormone present in the normal subject, and a concentration of triiodothyronine which is higher than the urinary concentration of triiodothyronine in the normal subject, diagnoses hyperthyroidism in the subject.

42. The method of claim 41, wherein step (b) comprises:
(1) contacting an agent capable of binding to thyroid stimulating hormone with the urine sample so as to bind thyroid stimulating hormone which is present in the sample to the agent;
(2) removing unbound urine sample;
(3) contacting the bound thyroid stimulating hormone with a detectable agent capable of binding to thyroid stimulating hormone so as to bind the detectable agent to the bound thyroid stimulating hormone;
(4) removing unbound detectable agent; and
(5) determining the amount of detectable agent which is bound to the thyroid stimulating hormone, thereby determining the amount of thyroid stimulating hormone in the urine sample.

43. The method of claim 42, wherein the agent capable of binding to thyroid stimulating hormone of step (1) is an antibody which binds to thyroid stimulating hormone.

44. The method of claim 42, wherein the agent capable of binding to thyroid stimulating hormone of step (1) is a receptor which binds to thyroid stimulating hormone.

45. The method of claim 42, wherein the detectable agent is an antibody which binds to an epitope on thyroid stimulating hormone which differs from the epitope to which the agent of step (1) binds.

46. The method of claim 42, wherein the detectable agent is labeled with a detectable marker.

47. The method of claim 41, wherein step (b) comprises:
(1) contacting an agent capable of binding to triiodothyronine with a pre-determined amount of detectable triiodothyronine and the urine sample, so as to form a complex between the agent and (i) the detectable triiodothyronine or (ii) the triiodothyronine present in the urine sample;
(2) determining the amount of detectable triiodothyronine which is bound to the agent, wherein the difference between the predetermined amount of detectable triiodothyronine and the amount of detectable triiodothyronine which is bound indicates the amount of triiodothyronine present in the urine sample.

48. The method of claim 41, wherein step (b) comprises:
(1) contacting an agent capable of binding to triiodothyronine with a pre-determined amount of detectable triiodothyronine and the urine sample, so as to form a complex between the agent and (i) the detectable triiodothyronine or (ii) the triiodothyronine present in the urine sample;
(2) determining the amount of detectable triiodothyronine which is not bound to the agent, thereby determining the amount of triiodothyronine present in the urine sample.

49. The method of claim 47 or 48, wherein the agent capable of binding to triiodothyronine of step (1) is an antibody which is capable of binding to triiodothyronine.

50. The method of claim 47 or 48, wherein the agent capable of binding to triiodothyronine of step (1) is a triiodothyronine receptor.

51. The method of claim 47 or 48, wherein the detectable triiodothyronine is labeled with a detectable marker.

52. The method of claim 41, wherein a concentration of thyroid stimulating hormone greater than 0.35 μIU/ml and a concentration of triiodothyronine greater then 1.5 ng/ml diagnoses hypothyroidism in the subject.

53. The method of claim 41, wherein a concentration of thyroid stimulating hormone less than 0.04 μIU/ml and a concentration of triiodothyronine less than 0.3 ng/ml diagnoses hyperthyroidism in the subject.

54. A method of diagnosing a thyroid condition in a subject which comprises:
a) obtaining a suitable urine sample from the subject;
b) determining the concentration of thyroid stimulating hormone and the concentration of thyroxine in the sample by a method which is not a radioimmunoassay;
c) comparing the concentration of thyroid stimulating hormone with a urinary concentration of thyroid stimulating hormone in a normal subject and comparing the concentration of thyroxine with a urinary concentration of thyroxine in a normal subject; wherein
i) a concentration of thyroid stimulating hormone which is higher than the urinary concentration of thyroid stimulating hormone in a normal subject, and a concentration of thyroxine which is lower than the urinary concentration of thyroxine in a normal subject, diagnoses hypothyroidism in the subject; and
ii) a concentration of thyroid stimulating hormone which is lower than the urinary concentration of thyroid stimulating hormone in a normal subject, and a concentration of thyroxine which is higher than the urinary concentration of thyroxine in a normal subject, diagnoses hyperthyroidism in the subject.

55. The method of claim 53, wherein step (b) comprises:
(1) contacting an agent capable of binding to thyroid stimulating hormone with the urine sample so as to bind thyroid stimulating hormone which is present in the sample to the agent;
(2) removing unbound urine sample;
(3) contacting the bound thyroid stimulating hormone with a detectable agent capable of binding to thyroid stimulating hormone so as to bind the detectable agent to the bound thyroid stimulating hormone;
(4) removing unbound detectable agent; and
(5) determining the amount of detectable agent which is bound to the thyroid stimulating hormone, thereby determining the amount of thyroid stimulating hormone in the urine sample.

56. The method of claim 55, wherein the agent capable of binding to thyroid stimulating hormone of step (1) is an antibody which binds to thyroid stimulating hormone.

57. The method of claim 55, wherein the detectable agent is an antibody which binds to an epitope on thyroid stimulating hormone which differs from the epitope to which the agent of step (1) binds.

58. The method of claim 55, wherein the agent capable of binding to thyroid stimulating hormone of step (1) is a receptor which binds to thyroid stimulating hormone.

59. The method of claim 55, wherein the detectable agent is labeled with a detectable marker.

60. The method of claim 54, wherein step (b) comprises:
(1) contacting an agent capable of binding to thyroxine with a pre-determined amount of detectable thyroxine and the urine sample, so as to form a complex between the agent and (i) the detectable thyroxine or (ii) the thyroxine present in the urine sample;
(2) determining the amount of detectable thyroxine which is bound to the agent, wherein the difference between the pre-determined amount of detectable thyroxine and the amount of detectable thyroxine which is bound indicates the amount of thyroxine present in the urine sample.

61. The method of claim 54, wherein step (b) comprises:
(1) contacting an agent capable of binding to thyroxine with a pre-determined amount of detectable thyroxine and the urine sample, so as to form a complex between the agent and (i) the detectable thyroxine or (ii) the thyroxine present in the urine sample;
(2) determining the amount of detectable thyroxine which is not bound to the agent, thereby determining the amount of thyroxine present in the urine sample.

62. The method of claim 60 or 61, wherein the agent of step (1) which is capable of binding to thyroxine is an antibody.

63. The method of claim 60 or 61, wherein the agent of step (1) which is capable of binding to thyroxine is a thyroxine receptor.

64. The method of claim 60 or 61, wherein the detectable thyroxine is labeled with a detectable marker.

65. The method of claim 54, wherein a concentration of thyroid stimulating hormone greater than 0.35 μIU/ml and a concentration of thyroxine greater then 1.5 ng/ml diagnoses hypothyroidism in the subject.

66. The method of claim 54, wherein a concentration of thyroid stimulating hormone less than 0.04 μIU/ml and a concentration of thyroxine less than 0.3 ng/ml diagnoses hyperthyroidism in the subject.

67. A method of determining whether a subject being treated with thyroxine is receiving a proper dosage of thyroxine which comprises:
a) obtaining a suitable urine sample from the subject;
b) determining the concentration of thyroid stimulating hormone in the sample by a method which is not a radioimmunoassay; and
c) comparing the concentration of thyroid stimulating hormone with a urinary concentration of thyroid stimulating hormone in a normal subject;
wherein a concentration of thyroid stimulating hormone which is higher or lower than the urinary concentration of thyroid stimulating hormone in a normal subject indicates that the subject is not receiving the proper dosage of thyroxine.

68. The method of claim 67, wherein step (b) comprises:
(1) contacting an agent capable of binding to thyroid stimulating hormone with the urine sample so as to bind thyroid stimulating hormone which is present in the sample to the agent;
(2) removing unbound urine sample;
(3) contacting the bound thyroid stimulating hormone with a detectable agent capable of binding to thyroid stimulating hormone so as to bind the detectable agent to the bound thyroid stimulating hormone;
(4) removing unbound detectable agent; and
(5) determining the amount of detectable agent which is bound to the thyroid stimulating hormone, thereby determining the amount of thyroid stimulating hormone in the urine sample.

69. The method of claim 68, wherein the agent of step (1) is an antibody which binds to thyroid stimulating hormone.

70. The method of claim 68, wherein the detectable agent is an antibody which binds to an epitope on thyroid stimulating hormone which differs from the epitope to which the agent of step (1) binds.

71. The method of claim 68, wherein the agent of step (1) which is capable of binding to thyroxine is a thyroid stimulating hormone receptor.

72. The method of claim 68, wherein the detectable agent is labeled with a detectable marker.

73. The method of claim 67, wherein a concentration higher than 0.35 μIU/ml or a concentration lower than 0.04 μIU/ml indicates that the subject is not receiving the proper dosage of thyroxine.

74. A method of determining whether a subject being treated with thyroxine is receiving a proper dosage of thyroxine which comprises:
a) obtaining a suitable urine sample from the subject;
b) determining the concentration of triiodothyronine in the sample by a method which is not a radioimmunoassay; and
c) comparing the concentration of triiodothyronine with a urinary concentration of triiodothyronine in a normal subject;
wherein a concentration of triiodothyronine which is higher or lower than the urinary concentration of triiodothyronine in a normal subject indicates that the subject is not receiving the proper dosage of thyroxine.

75. The method of claim 74, wherein step (b) comprises:
(1) contacting an agent capable of binding to triiodothyronine with a pre-determined amount of detectable triiodothyronine and the urine sample, so as to form a complex between the agent and (i) the detectable triiodothyronine or (ii) the triiodothyronine present in the urine sample; and
(2) determining the amount of detectable triiodothyronine which is bound to the agent, wherein the difference between the pre-determined amount of detectable triiodothyronine and the amount of detectable triiodothyronine which is bound indicates the amount of triiodothyronine present in the urine sample.

76. The method of claim 74, wherein step (b) comprises:
(1) contacting an agent capable of binding to triiodothyronine with a pre-determined amount of detectable triiodothyronine and the urine sample, so as to form a complex between the agent and (i) the detectable triiodothyronine or (ii) the triiodothyronine present in the urine sample;
(2) determining the amount of detectable triiodothyronine which is not bound to the agent, thereby determining the amount of triiodothyronine present in the urine sample.

77. The method of claim 75 or 76, wherein the agent of step (1) which is capable of binding to triiodothyronine is an antibody.

78. The method of claim 75 or 76, wherein the agent of step (1) which is capable of binding to triiodothyronine is a triiodothyronine receptor.

79. The method of claim 75 or 76, wherein the detectable triiodothyronine is labeled with a detectable marker.

80. The method of claim 74, wherein a concentration lower than 0.3 ng/ml or a concentration higher than 1.5 ng/ml indicates that the subject is not receiving the proper dosage of thyroxine.

81. A method of determining whether a subject being treated with thyroxine is receiving a proper dosage of thyroxine which comprises:
a) obtaining a suitable urine sample from the subject;
b) determining the concentration of thyroxine in the sample by a method which is not a radioimmunoassay;
c) comparing the concentration of thyroxine with a urinary concentration of thyroxine in a normal subject;
wherein a concentration of thyroxine which is higher or lower than the urinary concentration of thyroxine in a normal subject indicates that the subject is not receiving the proper dosage of thyroxine.

82. The method of claim 81, wherein step (b) comprises:
(1) contacting an agent capable of binding to thyroxine with a pre-determined amount of detectable thyroxine and the urine sample, so as to form a complex between the agent and (i) the detectable thyroxine or (ii) the thyroxine present in the urine sample;
(2) determining the amount of detectable thyroxine which is bound to the agent, wherein the difference between the pre-determined amount of detectable thyroxine and the amount of detectable thyroxine which is bound indicates the amount of thyroxine present in the urine sample.

83. The method of claim 81, wherein step (b) comprises:
(1) contacting an agent capable of binding to thyroxine with a pre-determined amount of detectable thyroxine and the urine sample, so as to form a complex between the agent and (i) the detectable thyroxine or (ii) the thyroxine present in the urine sample;
(2) determining the amount of detectable thyroxine which is not bound to the agent, thereby determining the amount of thyroxine present in the urine sample.

84. The method of claim 82 or 83, wherein the agent of step (1) which is capable of binding to thyroxine is an antibody.

85. The method of claim 82 or 83, wherein the agent of step (1) which is capable of binding to thyroxine is a thyroxine receptor.

86. The method of claim 82 or 83, wherein the detectable thyroxine is labeled with a detectable marker.

87. The method of claim 81, wherein a concentration lower than 0.3 ng/ml or a concentration higher than 1.5 ng/ml indicates that the subject is not receiving the proper dosage of thyroxine.

88. A method of monitoring a subject being treated with thyroxine and ensuring that the subject receives the proper dosage of thyroxine which comprises:
(a) determining whether the subject is receiving the proper dosage of thyroxine the method of any one of claims 67, 74 and 81;
(b) adjusting the dosage if it is determined that the subject is not receiving the proper dosage;
(c) repeating steps (a) through (b) throughout the course of the treatment;
thereby monitoring the subject being treated with thyroxine and ensuring that the subject receives the proper dosage of thyroxine.

89. The method of any one of claims 2, 10, 11, 18, 19, 26, 27, 34, 35, 42, 43, 47, 48, 55, 60, 61, 68, 75, 76, 82 and 83, wherein the agent of step (1) is immobilized.

90. The method of claim 89, wherein the agent is immobilized on a gold particle, a latex particle, a magnetic particle or other solid phase.

91. The method of any one of claims 1, 9, 17, 25, 33, 41, 54, 67, 74 and 81, wherein the urine sample is concentrated.

92. The method of any one of claims 1, 9, 17, 25, 33, 41, 54, 67, 74 and 81, wherein the urine sample is not concentrated.

93. The method of any one of claims 6, 14, 22, 30, 38, 46, 51, 59, 64, 72, 79 and 86, wherein the detectable marker is a colorimetric, a luminescent, or a fluorescent marker.
